(19) United States
(12) Patent Application Publication
(10) Pub. No.: US 2013/0230571 A1
(43) Pub. Date: Sep. 5, 2013

(54) MACROCYCLIC LACTONE COMPOUNDS AND METHODS FOR THEIR USE

(71) Applicants: John Yan, Los Gatos, CA (US); Xiaoxia Zheng, Milpitas, CA (US); Vinayak Bhat, Cupertino, CA (US)

(72) Inventors: John Yan, Los Gatos, CA (US); Xiaoxia Zheng, Milpitas, CA (US); Vinayak Bhat, Cupertino, CA (US)

(73) Assignee: Elixir Medical Corporation, Sunnyvale, CA (US)

(21) Appl. No.: 13/878,748

(22) PCT Filed: Oct. 3, 2011
(86) PCT No.: PCT/US11/54637

§ 371 Date: May 14, 2013

Related U.S. Application Data
(60) Provisional application No. 61/389,422, filed on Oct. 4, 2010.

(51) Int. Cl. A61L 31/16; A61L 31/08

(52) U.S. Cl. CPC A61L 31/16; A61L 31/08
USPC 424/400; 514/291; 424/133.1; 514/44 R; 514/171; 514/226.5

(57) ABSTRACT

The present disclosure provides a device for intracorporeal use which comprises an implant or a temporary device and at least one source of myolimus or a derivative thereof. The present disclosure also provides a method of inhibiting cell proliferation, inflammation or cytokine production by systemic or local administration of a therapeutically effective amount of myolimus or a derivative thereof. Further included in the present disclosure is a method of treating an ophthalmic condition or disease by administering a therapeutically effective amount of myolimus or a derivative thereof.

### CROSS-REFERENCES TO RELATED APPLICATIONS

The present application claims priority to U.S. Provisional Patent Application No. 61/389,422, filed on Oct. 4, 2010, which is incorporated herein in its entirety for all purposes.

### FIELD OF THE DISCLOSURE

The present disclosure relates to macrocylic lactones, myolimus and derivatives thereof and their use in therapeutic applications.

### BACKGROUND

Rapamycin (Sirolimus) is a 31-member natural macrocyclic lactone [C51H79N1O13; MWt=914.2] produced by Streptomyces hygroscopicus and found in the 1970s (U.S. Pat. No. 3,929,992; 3,993,749). Rapamycin (structure shown below) was approved by the Food and Drug Administration (FDA) for the prophylaxis of renal transplant rejection in 1999.

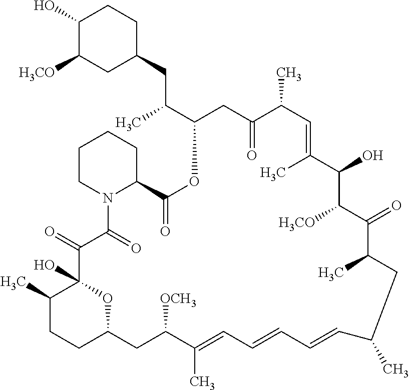

Rapamycin resembles tacrolimus (binds to the same intracellular binding protein or immunophilin known as FKBP-12) but differs in its mechanism of action. Whereas tacrolimus and cyclosporine inhibit T-cell activation by blocking lymphokine (e.g., IL2) gene transcription, sirolimus inhibits T-cell activation and T lymphocyte proliferation by binding to mammalian target of rapamycin (mTOR). Rapamycin can act in synergy with cyclosporine or tacrolimus in suppressing the immune system.

Rapamycin is also useful in preventing or treating systemic lupus erythematosus [U.S. Pat. No. 5,078,999], pulmonary inflammation [U.S. Pat. No. 5,080,899], insulin dependent diabetes mellitus [U.S. Pat. No. 5,321,009], skin disorders, such as psoriasis [U.S. Pat. No. 5,286,730], bowel disorders [U.S. Pat. No. 5,286,731], smooth muscle cell proliferation and intimal thickening following vascular injury [U.S. Pat. Nos. 5,288,711 and 5,516,781], adult T-cell leukemia/lymphoma [European Patent Application 525,960 A1], ocular inflammation [U.S. Pat. No. 5,387,589], malignant carcinomas [U.S. Pat. No. 5,206,018], cardiac inflammatory disease [U.S. Pat. No. 5,496,832], anemia [U.S. Pat. No. 5,561,138] and increase neurite outgrowth [Parker, E. M. et al, Neuropharmacology 39, 1913-1919, 2000].

Although rapamycin can be used to treat various disease conditions, the utility of the compound as a pharmaceutical drug has been limited by its very low and variable bioavailability and its high immunosuppressive potency and potential high toxicity. Also, rapamycin is only very slightly soluble in water. To overcome these problems, prodrugs and analogues of the compound have been synthesized. Water soluble prodrugs prepared by derivatizing rapamycin positions 31 and 42 (formerly positions 28 and 40) of the rapamycin structure to form glycinate, propionate, and pyrrolidino butyrate prodrugs have been described (U.S. Pat. No. 4,650,803). Some of the analogues of rapamycin described in the art include monoacyl and diacyl analogues (U.S. Pat. No. 4,316,885), acetal analogues (U.S. Pat. No. 5,151,413), silyl ethers (U.S. Pat. No. 5,120,842), hydroxyesters (U.S. Pat. No. 5,362,718), as well as alkyl, aryl, alkenyl, and alkynyl analogues (U.S. Pat. Nos. 5,665,772; 5,258,389; 6,384,046; WO 97/35575).

Prodrugs and analogues of rapamycin are synthesized by chemical synthesis, where additional synthetic steps are required to protect and deprotect certain positions. Analogues can also be synthesized biologically, where the Streptomyces strain is genetically modified to produce these analogues of rapamycin. The analogues need to maintain necessary positions for protein binding or other cellular interactions and not generate steric hindrance in order to preserve its activity. The safety of these analogues requires extensively testing by series of preclinical and clinical experimentations.

The present disclosure comprises novel use of macrocyclic lactones with at least some immunosuppressive, anti-proliferative, anti-fungal and anti-tumor properties for use in site specific applications.

### BRIEF SUMMARY OF THE DISCLOSURE

In one embodiment, the present disclosure provides a device for intracorporeal use, the device having an implant or a temporary device; and at least one source comprising a compound, wherein the compound is myolimus or a derivative thereof, and the amount of compound on or associated with the device is from about 10 microgram/cm² to about 400 microgram/cm².

In a second embodiment, the present disclosure provides a method of inhibiting cell proliferation in a subject in need thereof by local administration to the subject of a therapeutically effective amount of a compound myolimus, or a derivative thereof, thereby inhibiting cell proliferation.

In a third embodiment, the present disclosure provides a method of treating an ophthalmic condition or disease in a subject in need thereof, including administering to the subject a therapeutically effective amount of a compound myolimus, or a derivative thereof, thereby treating the ophthalmic condition or disease

### BRIEF DESCRIPTION OF THE DRAWINGS

FIG. 1 shows the potency of myolimus as compared to rapamycin at varying concentrations following exposure for 8 hours.

FIGS. 2(a) and (b) show the stent-based and tissue-based kinetics of a myolimus-eluting stent with a durable polymer.

FIGS. 3(a) and (b) show the stent-based and tissue-based kinetics of a myolimus-eluting stent with a bioabsorbable polymer.

FIG. 4 shows myolimus inhibiting production of MMP-9 as compared to rapamycin which increased the production of MMP-9.

FIG. 5 shows myolimus inhibiting the production of pro-inflammatory cytokine MCP-1 as compared to rapamycin which did not impact the production of MCP-1.

FIG. 6 shows an example of a stent configuration having an expandable structure.

### DETAILED DESCRIPTION OF THE DISCLOSURE

### I. Definitions

As used herein, the term “acid” refers to any chemical compound that, when dissolved in water, gives a solution with a pH less than 7.0. Acids are generally described as a compound which donates a hydrogen ion (H⁺) (Bronsted-Lowry) or as an electron-pair acceptor (Lewis acid). Acids useful in the present disclosure include, but are not limited to, HCl, H₂SO₄, HNO₃ and acetic acid. One of skill in the art will appreciate that other acids are useful in the present disclosure.

As used herein, “administering” refers to systemic or local administration or a combination thereof to a subject, such as oral administration, administration as a suppository, topical contact, parenteral, intravascular, intravenous, intraperitoneal, intramuscular, intralesional, intranasal, pulmonary, mucosal, transdermal, subcutaneous, intrathecal, intraocular and intravitreal administration, delivery from a temporary device such as catheters, balloons and porous balloons, delivery from an implant such as polymeric implants, metallic implants, drug-eluting stents, wraps, pumps such as osmotic pumps, contact lenses, and other routes and modes of administration. One of skill in the art will appreciate that other modes and methods of administering the compounds of the present disclosure are useful in the present disclosure.

As used herein, the term “associated with” refers to any form of association, including without limitation directly or indirectly being absorbed in, absorbed on, adhered to, adjacent to, attached to, bonded to, contained in, contained on, connected to, coupled to, dispersed in, disposed in, disposed on, entrapped in, incorporated in, and other forms of association.

As used herein, the term “alkoxy” refers to alkyl with the inclusion of an oxygen atom, for example, methoxy, ethoxy, etc. “Halo-substituted-alkoxy” is as defined for alkoxy where some or all of the hydrogen atoms are substituted with halogen atoms. For example, halo-substituted-alkoxy includes trifluoromethoxy, etc. One of skill in the art will appreciate that other alkoxy groups are useful in the present disclosure.

As used herein, the term “alkyl” refers to a straight or branched, saturated, aliphatic radical having the number of carbon atoms indicated. For example, C₁-C₆ alkyl includes, but is not limited to, methyl, ethyl, propyl, butyl, pentyl, hexyl, iso-propyl, iso-butyl, sec-butyl, tert-butyl, etc. One of skill in the art will appreciate that other alkyl groups are useful in the present disclosure.

As used herein, the term “hydroxyalkyl” refers to alkyl as defined above where at least one of the hydrogen atoms is substituted with a hydroxy group. For example, hydroxyalkyl includes hydroxy-methyl, hydroxy-ethyl (1- or 2-), hydroxy-propyl (1-, 2- or 3-), hydroxy-butyl (1-, 2-, 3- or 4-), hydroxy-pentyl (1-, 2-, 3-, 4- or 5-), hydroxy-hexyl (1-, 2-, 3-, 4-, 5- or 6-), 1,2-dihydroxyethyl, and the like. One of skill in the art will appreciate that other hydroxyalkyl groups are useful in the present disclosure.

As used herein, the term “body lumen” refers to the surface or lining or cavity of an artery, vein, capillary, or of an organ.

As used herein, the term “contacting” refers to the process of bringing into contact at least two distinct species such that they can interact. It should be appreciated, however, that the resulting reaction product can be produced directly from a reaction between the added reagents or from an intermediate from one or more of the added reagents which can be produced in the reaction mixture.

As used herein, the term “hydrate” refers to a compound that is complexed to at least one water molecule. The compounds of the present disclosure can be complexed with from 1 to 100 water molecules.

As used herein, the term “implant” refers to a nondegradable or degradable medical device inserted into a intracorporeal body in order to treat a condition. Implants include, but are not limited to, drug-eluting devices.

As used herein, the terms “inhibition”, “inhibits” and “inhibitor” refer to a compound that prohibits, reduces, diminishes or lessens, or to a method of prohibiting, reducing, diminishing or lessening a specific action or function.

As used herein, the term “intracorporeal” refers to an mammalian body.

As used herein, the term “isomer” refers to compounds of the present disclosure that possess asymmetric carbon atoms (optical centers) or double bonds, the racemates, diastereomers, enantiomers, geometric isomers, structural isomers and individual isomers are all intended to be encompassed within the scope of the present disclosure.

As used herein, the term “organ” refers to any organ of a mammal, such as, but not limited to, heart, lungs, brain, eye, stomach, spleen, bones, pancreas, kidneys, liver, intestines, uterus, colon, ovary, blood, skin, muscle, tissue, prostate, vascular (including arteries, veins and capillaries), spine, lymphatic system, pericardium, nervous system, cochlear, sinusus, mammary and bladder. One of skill in the art will appreciate that other organs are useful in the present disclosure.

As used herein, the term “peracid” refers to an acid in which an acidic —OH group has been replaced by an —OOH group. Peracids can be peroxy-carboxylic acids of the formula R—C(O)—OOH, where the R group can be groups such as H, alkyl, alkene or aryl. Peracids include, but are not limited to, peroxy-acetic acid and meta-chloro-peroxybenzoic acid (MCPBA). One of skill in the art will appreciate that other peracids are useful in the present disclosure.

As used herein, the term “peroxide” refers to a compound containing an oxygen-oxygen single bond. Examples of peroxides include, but are not limited to, hydrogen peroxide. One of skill in the art will appreciate that other peroxides are useful in the present disclosure.

As used herein, the term “pharmaceutically acceptable excipient” refers to a substance that aids the administration of an active agent to a subject. Pharmaceutical excipients useful in the present disclosure include, but are not limited to, polymers, solvents, antioxidants, binders, fillers, disintegrants, lubricants, coatings, sweeteners, flavors, stabilizers, colorants, metals, ceramics and semi-metals. See below for additional discussion of pharmaceutically acceptable excipients. One of skill in the art will recognize that other pharmaceutical excipients are useful in the present disclosure.

As used herein, the term “polymer” refers to a molecule composed of repeating structural units, or monomers, connected by chemical bonds. Polymers useful in the present disclosure are described below. One of skill in the art will appreciate that other polymers are useful in the present disclosure.

As used herein, the term “prodrug” refers to compounds which are capable of releasing the active agent of the methods of the present disclosure, when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of the active agents of the present disclosure include active agents wherein a hydroxy, amidino, guanidino, amino, carboxylic or a similar group is modified.

As used herein, the term “salt” refers to acid or base salts of the compounds used in the methods of the present disclosure. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present disclosure include salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds can be regenerated by contacting the salt with a base or acid and isolating the parent compound in the conventional manner. The parent form of the compound may differ from the various salt forms in certain physical properties, such as solubility in polar solvents, but otherwise the salts are equivalent to the parent form of the compound for the purposes of the present disclosure.

As used herein, the term “source” refers to a composition that includes at least one of a compound of the present disclosure, a therapeutic agent, and/or a pharmaceutically acceptable excipient. The device of the present disclosure can have at least one source. A source can include a compound of the present disclosure while another source can include a therapeutic agent. A source can have a compound and therapeutic agent and can be used to treat same or different indication. The device of the present disclosure can have at least one source on at least part of the device. The source on the device can coat at least part of the device, can be contained within a coating such as a polymer, can be contained within a reservoir, can be contained within a rate limiting barrier, can be contained within a micro-encapsulation, or one or more of the above.

As used herein, the term “subject” refers to animals such as mammals, including, but not limited to, primates (e.g., humans), cows, sheep, goats, horses, pigs, dogs, cats, rabbits, rats, mice and the like. In certain embodiments, the subject is a human.

As used herein, the term “therapeutic agent” refers to any agent, compound or biological molecule that has a therapeutic effect on the patient to whom the therapeutic agent is administered.

As used herein, the terms “therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques.

As used herein, the term “vascular prosthesis” refers to an implant for the circulatory system of a mammal.

### II. Compounds of the Present Disclosure

The present disclosure includes macrocyclic lactones, myolimus (also known as 32-deoxorapamycin and SAR943) and derivatives of myolimus, as described herein and described in WO 03/057218, which is incorporated herein in its entirety. Macrocyclic lactones, their salts, solvates, hydrates, N-oxides, isomers (including without limitation stereochemical isomers, conformational isomers, regiochemical isomers, structural isomers and ring isomers), tautomers, ring-opened congeners, analogues, derivatives, prodrugs and metabolites are collectively referred to as “macrocyclic lactones” in this disclosure, and compounds of the present disclosure encompass such macrocyclic lactones.

In some embodiments, the present disclosure encompasses macrocyclic lactones of compounds of formula (I):

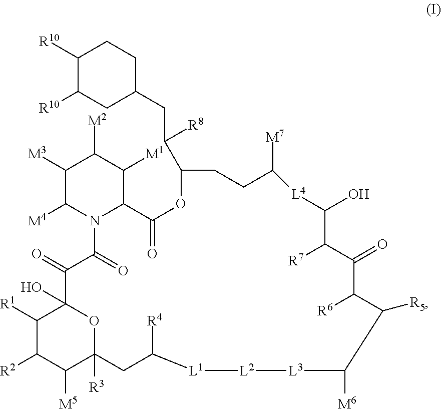

wherein:

R¹, R², R³, R⁵, R⁶, R⁸, M¹, M², M³, M⁴, M⁵, M⁶ and M⁷ are each independently H, —OH, C₁₋₆ alkyl or C₁₋₆ hydroxyalkyl;

R⁴, R⁷ and R⁹ are each independently H, —OH or C₁₋₆ alkoxy;

R¹⁰ is H, —OH, —OP(O)Me₂,

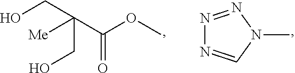

—O—(CH₂)ₙ—OH or —O—(CH₂)ₘ—O—(CH₂)ₒ—CH₃, wherein subscripts n and m are each independently from 2 to 8 and subscript o is from 1 to 6;

L¹ and L⁴ are each independently

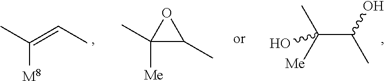

wherein each M⁸ is independently C₁₋₆ alkyl or C₁₋₆ hydroxyalkyl; and

L² and L³ are each independently

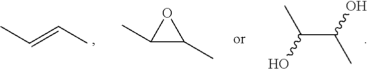

The compounds of the present disclosure include myolimus and its derivatives as described by the structure below

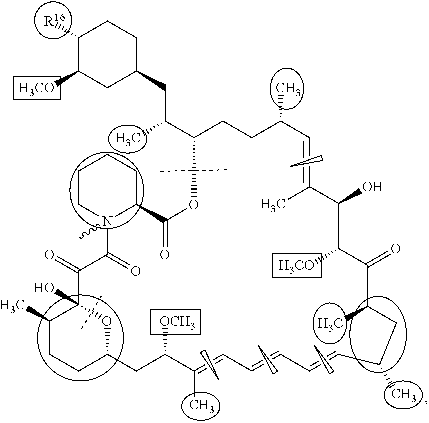

wherein squares represent positions that can undergo demethylation, or replacement with alkylhydroxy. Circles represent positions that can undergo substitution with a hydroxy group. Triangles represent positions that can undergo epoxidation or dihydroxylation. Curved lines represent potential N-oxidation position. Dashed lines represent potential positions for ring-opening, which may result in formation of, e.g., a carbonyl group at C-10 and/or a hydroxyl group at C-34. R¹⁰ is a member selected from the group consisting of H, —OH, —OP(O)Me₂,

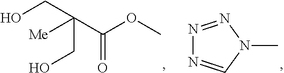

—O—(CH₂)ₙ—OH and —O—(CH₂)ₘ—O—(CH₂)ₒ—CH₃, wherein subscripts n and m are each independently from 2 to 8 and subscript o is from 1 to 6.

In some embodiments, R¹⁰ can be hydroxy, hydroxyalkyl, hydroxyalkylene, tetrazolyl, phosphinates, phosphates, ethers such as —O—R¹⁵ and —O—R¹⁵—O—R¹⁶, where R¹⁵ and R¹⁶ each independently are linear or branched C₁-C₆ alkyl, and propionic acid derivatives such as dimethyloylpropionic acid.

In some other embodiments, the compound of the present disclosure is myolimus, or 32-deoxo-rapamycin, which has the following structure:

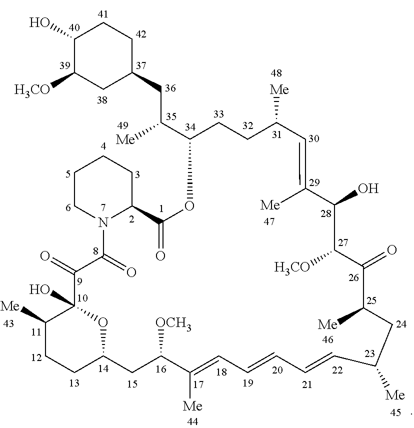

In certain embodiments, the present disclosure encompasses isomers, derivatives and analogs of myolimus. Non-limiting examples of isomers, derivatives and analogs of myolimus include:
N7-oxide-myolimus;
2-hydroxy-myolimus;
3-hydroxy-myolimus;
4-hydroxy-myolimus;
5-hydroxy-myolimus;
6-hydroxy-myolimus;
11-hydroxy-myolimus;
12-hydroxy-myolimus;
13-hydroxy-myolimus;
14-hydroxy-myolimus;
23-hydroxy-myolimus;
24-hydroxy-myolimus;
25-hydroxy-myolimus;
31-hydroxy-myolimus;
35-hydroxy-myolimus;
43-hydroxy-myolimus (or 11-hydroxymethyl-myolimus);
44-hydroxy-myolimus (or 17-hydroxymethyl-myolimus);
45-hydroxy-myolimus (or 23-hydroxymethyl-myolimus);
46-hydroxy-myolimus (or 25-hydroxymethyl-myolimus);
47-hydroxy-myolimus (or 29-hydroxymethyl-myolimus);
48-hydroxy-myolimus (or 31-hydroxymethyl-myolimus);
49-hydroxy-myolimus (or 35-hydroxymethyl-myolimus);
16-O-desmethyl-myolimus;
27-O-desmethyl-myolimus;
39-O-desmethyl-myolimus;
16-desmethoxy-myolimus;
27-desmethoxy-myolimus;
39-desmethoxy-myolimus;
17,18-epoxide-myolimus;
19,20-epoxide-myolimus;
21,22-epoxide-myolimus;
29,30-epoxide-myolimus;
17,18-dihydroxy-myolimus;
19,20-dihydroxy-myolimus;
21,22-dihydroxy-myolimus;
29,30-dihydroxy-myolimus;
myolimus having the hemi-ketal pyranyl ring opened at C-10, and having hydroxyl groups at C-10 and C-14;
myolimus having the hemi-ketal pyranyl ring opened at C-10, and having a carbonyl group at C-10 and a hydroxyl group at C-14;
myolimus having the hemi-ketal pyranyl ring opened at C-10, and having an oxepanyl ring that includes the six carbon atoms C-9 to C-14, a hydroxyl group at C-9 and a carbonyl group at C-10;
myolimus ring-opened at C-1, and having a carboxyl or ester group at C-2 and a hydroxyl group at C-34;
myolimus ring-opened at C-34, and having a carboxyl or ester group at C-2 and an alkenyl group at C33-C34;
myolimus ring-opened at C-34, and having a carboxyl or ester group at C-2 and an epoxide group at C33-C34;
myolimus ring-opened at C-34, and having a carboxyl or ester group at C-2 and a dihydroxy group at C33-C34;
myolimus ring-opened at C-34, and having a carboxyl or ester group at C-2 and methylene groups at C-33 and C-34;
2-epi-myolimus;
10-epi-myolimus;
11-epi-myolimus;
14-epi-myolimus;
16-epi-myolimus;
23-epi-myolimus;
25-epi-myolimus;
27-epi-myolimus;
28-epi-myolimus;
31-epi-myolimus;
34-epi-myolimus;
35-epi-myolimus;
37-epi-myolimus;
39-epi-myolimus; and
40-epi-myolimus.

The present disclosure also encompasses isomers, derivatives and analogs of myolimus having any combinations of two or more modifications to myolimus described herein, including without limitation:
16,27-O-bis(desmethyl)-myolimus;
16,39-O-bis(desmethyl)-myolimus;
27,39-O-bis(desmethyl)-myolimus;
16,27,39-O-tris(desmethyl)-myolimus;
16-O-desmethyl-N7-oxide-myolimus;
16-O-desmethyl-2-hydroxy-myolimus;
16-O-desmethyl-3-hydroxy-myolimus;
16-O-desmethyl-4-hydroxy-myolimus;
16-O-desmethyl-5-hydroxy-myolimus;
16-O-desmethyl-6-hydroxy-myolimus;
16-O-desmethyl-11-hydroxy-myolimus;
16-O-desmethyl-12-hydroxy-myolimus;
16-O-desmethyl-13-hydroxy-myolimus;
16-O-desmethyl-14-hydroxy-myolimus;
16-O-desmethyl-23-hydroxy-myolimus;
16-O-desmethyl-24-hydroxy-myolimus;
16-O-desmethyl-25-hydroxy-myolimus;
16-O-desmethyl-31-hydroxy-myolimus;
16-O-desmethyl-35-hydroxy-myolimus;
16-O-desmethyl-43-hydroxy-myolimus;
16-O-desmethyl-44-hydroxy-myolimus;
16-O-desmethyl-45-hydroxy-myolimus;
16-O-desmethyl-46-hydroxy-myolimus;
16-O-desmethyl-47-hydroxy-myolimus;
16-O-desmethyl-48-hydroxy-myolimus;
16-O-desmethyl-49-hydroxy-myolimus;
27-O-desmethyl-N7-oxide-myolimus;
27-O-desmethyl-2-hydroxy-myolimus;
27-O-desmethyl-3-hydroxy-myolimus;
27-O-desmethyl-4-hydroxy-myolimus;
27-O-desmethyl-5-hydroxy-myolimus;
27-O-desmethyl-6-hydroxy-myolimus;
27-O-desmethyl-11-hydroxy-myolimus;
27-O-desmethyl-12-hydroxy-myolimus;
27-O-desmethyl-13-hydroxy-myolimus;
27-O-desmethyl-14-hydroxy-myolimus;
27-O-desmethyl-23-hydroxy-myolimus;
27-O-desmethyl-24-hydroxy-myolimus;
27-O-desmethyl-25-hydroxy-myolimus;
27-O-desmethyl-31-hydroxy-myolimus;
27-O-desmethyl-35-hydroxy-myolimus;
27-O-desmethyl-43-hydroxy-myolimus;
27-O-desmethyl-44-hydroxy-myolimus;
27-O-desmethyl-45-hydroxy-myolimus;
27-O-desmethyl-46-hydroxy-myolimus;
27-O-desmethyl-47-hydroxy-myolimus;
27-O-desmethyl-48-hydroxy-myolimus;
27-O-desmethyl-49-hydroxy-myolimus;
39-O-desmethyl-N7-oxide-myolimus;
39-O-desmethyl-2-hydroxy-myolimus;
39-O-desmethyl-3-hydroxy-myolimus;
39-O-desmethyl-4-hydroxy-myolimus;
39-O-desmethyl-5-hydroxy-myolimus;
39-O-desmethyl-6-hydroxy-myolimus;
39-O-desmethyl-11-hydroxy-myolimus;
39-O-desmethyl-12-hydroxy-myolimus;
39-O-desmethyl-13-hydroxy-myolimus;
39-O-desmethyl-14-hydroxy-myolimus;
39-O-desmethyl-23-hydroxy-myolimus;
39-O-desmethyl-24-hydroxy-myolimus;
39-O-desmethyl-25-hydroxy-myolimus;
39-O-desmethyl-31-hydroxy-myolimus;
39-O-desmethyl-35-hydroxy-myolimus;
39-O-desmethyl-43-hydroxy-myolimus;
39-O-desmethyl-44-hydroxy-myolimus;
39-O-desmethyl-45-hydroxy-myolimus;
39-O-desmethyl-46-hydroxy-myolimus;
39-O-desmethyl-47-hydroxy-myolimus;
39-O-desmethyl-48-hydroxy-myolimus;
39-O-desmethyl-49-hydroxy-myolimus;
17,18-19,20-bis(epoxide)-myolimus;
17,18-29,30-bis(epoxide)-myolimus;
17,18-19,20-21,22-tris(epoxide)-myolimus;
19,20-21,22-29,30-tris(epoxide)-myolimus;
16-O-desmethyl-17,18-19,20-bis(epoxide)-myolimus;
16-O-desmethyl-17,18-29,30-bis(epoxide)-myolimus;
27-O-desmethyl-17,18-19,20-bis(epoxide)-myolimus;
27-O-desmethyl-17,18-29,30-bis(epoxide)-myolimus;
39-O-desmethyl-17,18-19,20-bis(epoxide)-myolimus;
39-O-desmethyl-17,18-29,30-bis(epoxide)-myolimus;
16-O-desmethyl-17,18-19,20-21,22-tris(epoxide)-myolimus;
16-O-desmethyl-19,20-21,22-29,30-tris(epoxide)-myolimus;
27-O-desmethyl-17,18-19,20-21,22-tris(epoxide)-myolimus;
27-O-desmethyl-19,20-21,22-29,30-tris(epoxide)-myolimus;
39-O-desmethyl-17,18-19,20-21,22-tris(epoxide)-myolimus;
39-O-desmethyl-19,20-21,22-29,30-tris(epoxide)-myolimus;
16,27-O-bis(desmethyl)-17,18-19,20-21,22-tris(epoxide)-myolimus;
16,27-O-bis(desmethyl)-19,20-21,22-29,30-tris(epoxide)-myolimus;
16,39-O-bis(desmethyl)-17,18-19,20-21,22-tris(epoxide)-myolimus;
16,39-O-bis(desmethyl)-19,20-21,22-29,30-tris(epoxide)-myolimus;
27,39-O-bis(desmethyl)-17,18-19,20-21,22-tris(epoxide)-myolimus;
27,39-O-bis(desmethyl)-19,20-21,22-29,30-tris(epoxide)-myolimus;
16-O-desmethyl-24-hydroxy-17,18-19,20-bis(epoxide)-myolimus;
16-O-desmethyl-24-hydroxy-17,18-29,30-bis(epoxide)-myolimus;
27-O-desmethyl-24-hydroxy-17,18-19,20-bis(epoxide)-myolimus;
27-O-desmethyl-24-hydroxy-17,18-29,30-bis(epoxide)-myolimus;
39-O-desmethyl-24-hydroxy-17,18-19,20-bis(epoxide)-myolimus;
39-O-desmethyl-24-hydroxy-17,18-29,30-bis(epoxide)-myolimus;
12-hydroxy-myolimus having the hemi-ketal pyranyl ring opened at C-10, and having hydroxyl groups at C-10 and C-14;
12-hydroxy-myolimus having the hemi-ketal pyranyl ring opened at C-10, and having a carbonyl group at C-10 and a hydroxyl group at C-14; and
isomers, derivatives and analogs of myolimus having hydroxyl groups at two or more of any of the positions described herein, including without limitation 12,24-dihydroxy-myolimus.

In further embodiments, the present disclosure encompasses compounds that have any of the R¹⁰ groups at C-40 described herein and also have any of the modifications or combinations of modifications of any of the myolimus isomers, derivatives and analogs described herein.

This disclosure further covers compositions comprising salts, solvates, hydrates, N-oxides, isomers, tautomers, ring-opened congeners, analogs, derivatives, prodrugs and metabolites of compounds of the present disclosure. In some embodiments, such compositions further comprise one or more pharmaceutically acceptable excipients. The disclosure also covers compounds with different polymorphic forms.

There have been different numbering schemes proposed for macrocyclic lactones. To avoid confusion, when specific macrocyclic lactones are named herein, the names are given with reference to macrocyclic lactone using the numbering scheme of the above chemical formula. This disclosure also covers all the macrocyclic lactones which have different name due to a different numbering scheme if the same functional group exists in the same location within the chemical structure. For example, 39-O-demethyl macrocyclic lactone is the same compound as 41-O-demethyl macrocyclic lactone and 16-O-demethyl macrocyclic lactone is the same compound as 7-O-demethyl macrocyclic lactone.

The compounds of the present disclosure can be prepared by a variety of methods. In some embodiments, the compounds of the present disclosure are synthesized biologically by genetically modifying the strains of organisms to produce the compounds of the present disclosure or by other means.

In another embodiment the compounds of the present disclosure are prepared using chemical synthesis.

The compounds of the present disclosure are optionally deuterated.

### III. Delivery of the Compounds of the Present Disclosure

The compounds of the present disclosure can be administered in any appropriate manner. In some embodiments, the compounds are administered intramuscularly, intraperitoneally, subcutaneously, pulmonarily, mucosally, transdermally, intravascularly, intraocularly or intravitreally through the eye, and others. In other embodiments, the compounds are administered site specifically through temporary or permanent drug delivery means such as a catheter or an implant or a combination of systemic and site specific means. Examples include, but are not limited to catheter, stent, wrap, pump, shunt or other temporary or permanent drug delivery means.

A. Device

In some embodiments, the present disclosure provides a device for intracorporeal use, the device includes an implant or a temporary device; and at least one source comprising a compound, wherein the compound is myolimus or a derivative thereof, and the amount of compound on or associated with the device is from about 10 microgram/cm² to about 400 microgram/cm².

In other embodiments, the present disclosure provides a device configured to release the compound to a body lumen or organ within an intracorporeal body to inhibit cell proliferation. In a further embodiment, the device is configured to release the compound to a body lumen or organ within an intracorporeal body to inhibit smooth muscle cell proliferation or inflammation.

The devices of the present disclosure can be delivered to a body lumen, outside a body lumen, adjacent to a body lumen, or proximal or distal to a body lumen, as well as to an organ, vessel, conduit, muscle, nerve, tissue mass or bone.

In another embodiment of the present disclosure the drug delivery means is a device such as an implant including graft implants, vascular implants, non-vascular implants, implantable luminal prostheses, wound closure implants, drug delivery implants, sutures, biologic delivery implants, urinary tract implants, inter-uterine implants, organ implants, ophthalmic implants, contact lenses, bone implants including bone plates, bone screws, dental implants, spinal disks, wraps such as vascular wraps or the like.

When the device is configured for treatment of ophthalmic conditions or diseases, the implant of the present disclosure can be implanted intraocularly or intravitreally by an intervention procedure. Such implants can be non-biodegradable, biodegradable, removable or permanent. In other embodiments, implants can be placed in the duct, such as the tear duct. In still other embodiments, implants can be placed adjacent to the ocular body, or intraocularly, adjacent to the vitreal body or intravitreally. One of skill in the art will appreciate that other locations are useful in the present disclosure.

The implant typically allows for one or more of the following: support, contain, hold together, affix, plug, open, close, maintain, deliver drug, deliver biologics for the prevention or treatment of disease conditions, such as for example proliferative diseases, restenosis, cardiovascular disease, inflammation, fibrosis, wound healing, cancer, neovascularization, aneurysm, diabetic disease, abdominal aortic aneurysm, hyper-calcemia, ophthalmic conditions, or others.

The implant of the present disclosure can be formed of metal, metal alloy, polymer, ceramic, semi-metal, nanocomposites or combination thereof. For example, an implant can be made from metal such as tantalum, iron, magnesium, molybdenum or others; from a degradable or non degradable metal alloy such as 316L stainless steel, carbon steel, magnesium alloy, NI-Ti, Co—Cr such as L605, MP35 or other; from a polymer that is degradable or non-degradable such as poly lactic acid, poly glycolic acid, poly esters, poly hydroxybutyrate, polyamide, poly (methyl methacrylate), poly(2-hydroxyethyl methacrylate) polymers (PHEMA), poly(dimethyl siloxane), poly(ethylene glycol), poly (ethylene glycol)-block-polyamino acid, hyaluronic acid, collagen, Poly peptide, polysaccharide or copolymers or others or blends of polymers; combination of metals and metals or metal alloys such as implant made from combination of layers of stainless steel and tantalum or others; nanocomposites such as nano carbon fibers or nano carbon tubules or others.

The implant of the present disclosure can take various shapes and forms such as a coil, a disk, a tube, a rod, a corrugated tube, a sheet, a chain, a screw, a scaffold, a microsphere, or others.

In another embodiment, the present disclosure provides a device wherein the implant comprises a vascular or other luminal prosthesis which is implanted in the lumen of a blood vessel or other body passage, such as a ureter, urethra, colon, trachea, bronchii, or the like. The device of the present disclosure can also be implanted outside of, or adjacent to, the body lumen. Such vascular and other luminal prostheses typically comprise an expandable tubular or other hollow structure, often referred to as a scaffold, where the scaffold is expanded in situ within the lumen of the blood vessel or other target body lumen to help maintain patency of the lumen. In specific embodiments, the vascular prosthesis comprises a stent or a graft, each of which usually comprises a scaffold or other open lattice structure. For example, a stent may comprise a bare scaffold or coated scaffold while a graft may comprise a covered scaffold, where the cover is a fabric or membrane which inhibits or prevents blood passage or tissue penetration though the open portions of the scaffold. In exemplary embodiments, the vascular prosthesis comprises a stent, typically a vascular stent, for intraluminal delivery to and deployment at a target location in a patient's vasculature.

In another embodiment, the present disclosure provides a device wherein the implant is a luminal prosthesis. In some embodiments, the luminal prosthesis comprises an expandable scaffold. In other embodiments, the vascular prosthesis comprises a stent or a graft. In still other embodiments, the luminal prosthesis is a vascular stent.

In another embodiment, the compounds of the present disclosure are associated with at least part of the implant. For example the compounds of the present disclosure can be incorporated within or on the implant, contained in openings, pores or cavities in or on the implant, contained in a (e.g., polymeric) matrix forming or disposed over at least a portion of the implant, contained in a (e.g., polymeric or metallic) coating disposed over at least a portion of the implant, or other.

In some embodiments, the present disclosure provides a device comprising a vascular prosthesis wherein the vascular prosthesis has a luminal and a tissue facing surface, and wherein the compound is associated with at least one of the luminal and tissue facing surfaces.

In a further embodiment, the compounds of the present disclosure are applied on all implant surfaces. In another embodiment, the compounds of the present disclosure are applied only to the abluminal or luminal surface. In yet another embodiment, the compounds of the present disclosure are applied only to higher stress or lower stress areas on the implant.

In another embodiment, the compounds of the present disclosure are contained within an erodible or non-erodible filament or filaments that are adjacent to the implant.

An example of a stent configuration for carrying a compound of the present disclosure is illustrated in FIG. 6 in a contracted state. The stent body is formed of multiple rings 110. The rings are formed of crowns 120 and struts 130 in a generally expandable undulating configurations such as, zigzag, sawtooth, sinusoidal wave or other. The body is joined by links or connectors 140. It is understood that the connectors may be of any length or shape, or may not be needed if the crowns are directly attached to each other. The stent has a typical contracted state diameter of between 0.25-4 mm, or between 0.7 to 1.5 mm, and a length of between 5 and 600 mm. In its expanded state, the stent diameter is typically at least twice and up to 10 times or more than that of the stent in its contracted state. For example, a stent with a contracted diameter of between 0.7 to 1.5 mm may expand radially to 2 to 10 mm or more.

Drug eluting stents with potent macrocyclic lactone compounds such as rapamycin (Cypher™) have resulted in late lumen loss in the range of approximately 0.01 mm to 0.2 mm at approximately 4 months to 12 months angiographic follow up. The late lumen loss with bare metal stents have ranged from approximately 0.70 mm to 1.2 mm for the same time period. Lower late lumen loss typically decreased the percent stenosis. However, significantly lower late lumen loss with drug eluting stents as compared to bare metal stents in some cases results in inadequate tissue coverage of the stent surface which potentially may increase incidence of late stent thrombosis.

In some embodiments, the present disclosure provides a device wherein the amount of at least one compound of the present disclosure associated with an implant is less than about 1 g/cm². In other embodiments, the amount of compound associated with the implant can range from about 1 nanogram/cm² to about 1000 microgram/cm², or from about 1 microgram/cm² to about 500 microgram/cm², or from about 10 microgram/cm² to about 400 microgram/cm². In still other embodiments, the amount of compound associated with the implant is less than about 1 mg. In yet other embodiments, the amount of compound associated with the implant is from about 1 μg to about 50 mg, or from about 100 μg to about 10 mg, or from about 100 μg to about 1 mg, or from about 200 μg to about 500 μg.

In a further embodiment, the present disclosure provides a device wherein the concentration of a compound of the present disclosure in tissue adjacent to the implant is from about 0.001 ng/gm tissue to about 1000 μg/gm tissue, or from about 1 ng/gm tissue to about 500 μg/gm tissue, or from about 100 ng/gm tissue to about 100 μg/gm tissue.

In another embodiment, the adjacent tissue comprises tissue less than 25 cm from the device. In other embodiments, the adjacent tissue comprises tissue less than about 10, 9, 8, 7, 6, 5, 4, 3, 2 or 1 centimeters from the device. In some other embodiments, the adjacent tissue comprises tissue less than about 9, 8, 7, 6, 5, 4, 3, 2 or 1 millimeters from the device. In still other embodiments, the adjacent tissue comprises tissue less than about 5 centimeters from the device. In yet other embodiments, the adjacent tissue comprises tissue less than about 1 centimeters from the device. In still yet other embodiments, the adjacent tissue comprises tissue less than about 5 millimeters from the device.

In another embodiment, the compounds of the present disclosure can be released from the implant over a period ranging from less than 5 minutes to 2 years, or from 3 days to 6 months, or from 1 week to 3 months. In other embodiments, the compounds of the present disclosure can be released from the implant over a period greater than 1 day, or greater than 2 weeks, or greater than 1 month. In another embodiment, the compounds of the present disclosure can require greater than 2 years to be fully released from the stent. In some embodiments, the amount of compound released over the interval described above is at least 25%. In other embodiments, the amount of compound released is at least 50%. In still other embodiments, the amount of compound released is at least 75%. In yet other embodiments, the amount of compound released can be at least 80, 85, 90, 91, 92, 93, 94, 95, 96, 97, 98 or 99%.

In a further embodiment, the present disclosure provides a device wherein at least 75% of the compound is released from the device in a period from about 1 day to about 2 years. In another embodiment, at least 90% of the compound is released from the device in a period from about 3 day to about 6 months. In still another embodiment, at least 90% of the compound is released from the device in a period from about 1 week to about 3 months.

When the compounds of the present disclosure are administered via site-specific implant through an intraocular or intravitreal body, the total dose, or dose per administration or dose over a certain period (e.g., daily, weekly, biweekly, monthly, etc.), of compound can vary from about 1 ug to about 5 mg, or from about 100 ug to about 1 mg, or from about 40 ug or 50 ug to about 500 ug, or from about 20 ug to about 200 ug. Following administration, the concentration of the compound of the present disclosure in the adjacent intraocular or intravitreal body can be from about 0.1 nM to about 500 mM, or from about 1 nM to about 1000 μM, or from about 10 nM to about 100 μM. One of skill in the art will appreciate that other doses and concentrations of the compounds of the present disclosure are useful.

The compounds of the present disclosure can be released from the implant via any means known in the art. In some embodiments, the implant releases the compound through active or passive means. In other embodiments, the implant releases the compound through osmotic pressure or diffusion. One of skill in the art will appreciate that other means of releasing the compound from the implant are useful in the present disclosure.

In some embodiments, the present disclosure provides a device that further includes an additional therapeutic agent, such as those described below. In some other embodiments, the additional therapeutic agent is released prior to, concurrent with, or subsequent to the release of the compound. In other embodiments, the compound is released from a first source and the additional therapeutic agent is released from a second source. In still other embodiments, the compound and the additional therapeutic agent are released from a single source. In yet other embodiments, the compounds and the additional therapeutic agent are released from the same source.

In some embodiments, the compound is released from the implant following an approximately first order release kinetics. In other embodiments, the compound is released from the implant following an approximately second order release kinetics. In yet other embodiments, the compound is released from the implant following a bust release followed by approximately first or second order release kinetics.

In some embodiments, the compound of the present disclosure can be released from a temporary device. In one embodiment, the temporary device is a catheter. The compound is delivered to the body lumen or organ via the catheter. In another embodiment, the temporary device is a porous balloon catheter or porous expandable member. The porous balloon catheter is delivered to the body lumen or organ and the catheter is inflated and the compound is delivered to the body lumen or organ through the porous balloon. In yet another embodiment, the temporary device is a coated balloon catheter. The compound is coated on the balloon with or without a polymer coating and the coated balloon catheter is delivered to the body lumen or organ and the catheter is inflated and the compound is delivered to the body lumen or organ contacting the coated balloon catheter.

B. Administration

The compounds of the present disclosure can be released from the implant at rates ranging from about 1 nanogram/cm²/day to about 1000 microgram/cm²/day, or from about 1 microgram/cm²/day to about 200 microgram/cm²/day, or from about 5 microgram/cm²/day to about 100 microgram/cm²/day.

In some embodiments, the present disclosure provides a device where the implant is a stent and the source coats the stent with myolimus at less than about 10 μg myolimus/mm stent, wherein the source includes poly(n-butylmethacrylate), such that the poly(n-butylmethacrylate) is present in a ratio of from about 1:5 to about 5:1 (w/w) to myolimus. In other embodiments, the implant is a stent and the source coats the stent with myolimus at less than about 10 μg myolimus/mm stent, wherein the source includes poly(L-lactide-co-glycolic acid), such that the poly(L-lactide-co-glycolic acid) is present in a ratio of from about 1:5 to about 5:1 (w/w) to myolimus. In some other embodiments, the implant is a stent and the source coats the stent with myolimus at less than about 10 μg myolimus/mm stent, wherein the source includes poly(ethylene carbonate), such that the poly(ethylene carbonate) is present in a ratio of from about 1:5 to about 5:1 (w/w) to myolimus. In still other embodiments, the implant is a stent and the source coats the stent with myolimus at less than about 10 μg myolimus/mm stent.

In yet other embodiments, the temporary device is a balloon and the source coats the balloon with myolimus at less than about 20 μg myolimus/mm balloon, wherein the source includes poly(ethylene-carbonate), such that the poly(ethylene-carbonate) is present in a ratio of from about 1:5 to about 5:1 (w/w) to myolimus. Other ratios and amounts of myolimus are useful in the devices of the present disclosure. In yet other embodiments, the temporary device is a balloon and the source coats the balloon with myolimus at less than about 20 μg myolimus/mm balloon. In yet other embodiments, the temporary device is a porous balloon catheter or porous expandable member and myolimus is delivered from the porous balloon or porous expandable member at concentrations of less than 1 mg/ml, where in the source includes myolimus and a solvent such as ethanol, DMSO, or other agents such as PEO, hydrating gels.

In other embodiments, the compounds of the present disclosure can be administered on a daily, intermittent or one-time dose basis. The total dose, or dose per administration or dose over a certain period (e.g., daily, weekly, biweekly, monthly, etc.), of a compound of the disclosure can range from about 0.1 mg to about 20 mg, or from about 0.5 mg to about 10 mg, or from about 1 mg to about 5 mg, or from about 1 μg to about 1 mg, or from about 100 μg to about 1 mg, or from about 40 μg or 50 μg to about 500 μg, or from about 20 μg to about 200 μg. One of skill in the art will appreciate that other doses are also useful in the present disclosure.

When the device of the present disclosure is configured for treatment of ophthalmic conditions or diseases, the compounds of the present disclosure can be administered through the eye as an eye drop or an injection on a daily, intermittent or one time dose basis. The total dose, or dose per administration or dose over a certain period (e.g., daily, weekly, biweekly, monthly, etc.), of a compound of the disclosure can range from about 0.1 μg to about 30 mg, or from about 10 μg to about 10 mg, or from about 1 μg to about 1 mg, or from about 100 μg to about 1 mg, or from about 40 μg or 50 μg to about 500 μg, or from about 20 μg to about 200 μg. Following administration, the concentration of the compound of the present disclosure in the adjacent intraocular or intravitreal body can be from about 0.1 nM to about 500 mM, or from about 1 nM to about 1000 μM, or from about 10 nM to about 100 μM. One of skill in the art will appreciate that other doses are also useful in the present disclosure.

C. Pharmaceutical Formulations

In some embodiments, the present disclosure provides a pharmaceutical composition wherein the pharmaceutically acceptable excipient is a member selected from the group consisting of a polymer, a solvent, an antioxidant, a binder, a filler, a disintegrant, a lubricant, a coating, a sweetener, a flavor, a stabilizer, a colorant, a metal, a ceramic and a semi-metal. In other embodiments, the pharmaceutically acceptable excipient is a polymer. In some other embodiments, the pharmaceutically acceptable excipient is other than a polymer.

The active ingredients of the present disclosure may be combined with pharmaceutically acceptable carriers, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, and dispensing agents, depending on the nature of the mode of administration and dosage forms. Such ingredients, including pharmaceutically acceptable carriers and excipients are described in the Handbook of Pharmaceutical Excipients, American Pharmaceutical Association (1986), incorporated herein by reference in its entirety. Examples of pharmaceutically acceptable carriers include water, ethanol, polyols, vegetable oils, fats, waxes polymers, including gel forming and non-gel forming polymers, and suitable mixtures thereof. Examples of excipients include starch, pregelatinized starch, Avicel, lactose, milk sugar, sodium citrate, calcium carbonate, dicalcium phosphate, and lake blend. Examples of disintegrating agents include starch, alginic acids, and certain complex silicates. Examples of lubricants include magnesium stearate, sodium lauryl sulphate, talc, as well as high molecular weight polyethylene glycols. One of skill in the art will appreciate that other different excepients can be used in formulations according to the present disclosure and the list provided herein is not exhaustive.

Suitable nondegradable or slow degrading polymer coatings include, but are not limited to, polyacrylamides, poly-N-vinylpyrrolidone, polydimethyl acrylamide, polymers and copolymers of 2-acrylamido-2-methyl-propanesulfonic acid, acrylic acids, methacrylic acids, polyurethanes, polyethylene imines, ethylene vinyl alcohol copolymers, silicones, C-flex, nylons, polyamides, polyimides, polytetrafluoroethylene (PTFE), parylene, parylast, poly(methyl methacrylate), poly(n-butyl methacrylate), poly(butyl methacrylate) copolymer or blended with poly(ethylene vinyl acetate), poly(methyl methacrylate), poly(2-hydroxyethyl methacrylate), poly(ethylene glycol methacrylate), polystyrene-b-isobutylene-b-styrene, copolymers of vinylidene fluoride and hexafluoropropylene, poly(vinyl chloride), poly(dimethyl siloxane), poly(ethylene vinyl acetate), polycarbonates, polyacrylamide gels, and the like, including other synthetic and natural polymeric substances; mixtures, copolymers, and combinations thereof.

Suitable biodegradable polymer coatings include, but are not limited to, polyesters, aliphatic polyesters, poly(lactic acid), polylactides, poly(L-lactide), poly(D-lactide), poly(LD-lactide), polylactates, poly(glycolic acid), polyglycolates and copolymers, poly(lactide-co-glycolide), poly(L-lactide-co-glycolide), polydioxanone, poly(ethyl glutamate), poly(hydroxybutyrates), polyhydroxyvalerates and copolymers, polycaprolactone, poly(lactide-co-caprolactone), poly(L-lactide-co-ε-caprolactone), polyanhydrides, salicylate based polyanhydride esters, salicylic acid-co-adipic acid-co-salicylic acid, salicylic acid-co-polylactide anhydride-salicylic acid, poly(orthoesters), poly(ether esters), polyethylene glycols, poly(ethylene oxides), poly(trimethylene carbonate), polyethylenecarbonate, copolymers of poly(ethylene carbonate) and poly(trimethylene carbonate), poly(propylene carbonate), poly(iminocarbonates), starch based polymers, cellulose acetate butyrate, polyester amides, polyester amines, polycyanoacrylates, polyphosphazenes, poly(N-vinyl-2-pyrrolidone), polymaleic anhydride, hyaluronic acid (hyaluronate), phosphoryl choline, chondroitin sulfate, dermatan sulfate, carboxymethylcellulose, heparin sulfate, keratan sulfate, carboxymethylhydroxypropylcellulose, carboxymethylhydroxethylcellulose, cellulose sulfate, cellulose phosphate, carboxymethyl guar, carboxymethylhydroxypropyl guar, carboxymethylhydroxyethylguar, xanthan gum, carrageenan, anionic polysaccharides, anionic proteins and polypeptides, quaternary ammonium compounds including stearyl ammonium chloride and benzyl ammonium chloride, copolymers thereof including copolymers of poly(lactic acid) and poly(caprolactone), and mixtures, copolymers, ionic polymers, and combinations thereof.

Suitable natural coatings include: fibrin, albumin, collagen, gelatin, glycosoaminoglycans, oligosaccharides and polysaccharides, chondroitin, chondroitin sulphates, hypoxyapatite, phospholipids, phosphorylcholine, glycolipids, fatty acids, proteins, cellulose, and mixtures, copolymers, and combinations thereof.

Suitable non-polymeric coatings include metallic coatings such as tungsten, magnesium, cobalt, zinc, iron, bismuth, tantalum, gold, platinum, stainless steel such as 316L, 304, titanium alloys; ceramics coatings such as silicon oxide; semi-metals such as carbon, nanoporous coatings; and combination thereof.

In some embodiments, the pharmaceutically acceptable excipient is a polymer selected from the group consisting of polyurethanes, polyethylene imines, ethylene vinyl alcohol copolymers, silicones, C-flex, nylons, polyamides, polyimides, polytetrafluoroethylene (PTFE), parylene, parylast, poly(methacrylate), poly(vinyl chloride), poly(dimethyl siloxane), poly(ethylene vinyl acetate), polycarbonates, polyacrylamide gels, poly(methyl methacrylate), poly(n-butyl methacrylate), poly(butyl methacrylate) copolymer or blended with poly(ethylene vinyl acetate), poly(2-hydroxyethyl methacrylate), poly(ethylene glycol methacrylates), poly-styrene-b-isobutylene-b-styrene, copolymers of vinylidene fluoride and hexafluoropropylene, poly(ethylene carbonate), poly(L-lactide-glycolide) copolymers, poly(L-lactide-trimethylene carbonate) copolymers, poly(L-lactide), poly(DL-lactide), poly(L-lactide-co-ε-caprolactone), salicylate based polyanhydride esters, salicylic acid-co-adipic acid-co-salicylic acid, salicylic acid-co-polylactide anhydride-salicylic acid, and phosphoryl choline. In a further embodiment, the polymer can be poly(n-butylmethacrylate), poly(ethylene carbonate), or poly(L-lactide-glycolide) copolymer.

In other embodiments, the polymer is a durable polymer. Durable polymers are stable under physiological conditions, and include polymers such as poly(n-butyl methacrylate). In some other embodiments, the polymer is a bioabsorbable, bioerodable or degradable polymer. The terms bioabsorbable, bioerodable and biodegradable can be used interchangeably and mean that the polymers biodegrade under physiological conditions, such as by hydrolysis or via enzymatic biodegradation. Bioabsorable polymers include polymers such as poly(ethylene carbonate) and poly(L-lactide-glycolide) copolymers. The rate of biodegradation of a bioabsorable polymer can be from 2 weeks to 5 years. In some embodiments, the biodegradation of a bioerodable or degradable polymer can be from 3 months to 2 years, in other embodiments, from 6 months to 12 months.

The degradation of the polymer can also be measured in average mass loss per day. For example, the polymer can decompose with a average loss of mass from 0.05% to 3% per day. Alternatively, the average loss of mass can be from 0.1% to 0.75% per day. Furthermore, the average loss of mass can be from 0.25% to 0.5% per day. Degradation of the polymer can also be measured by the average loss of volume per day, for example from 0.05% to 3% per day. Sometimes, the average loss of volume can be from 0.1% to 0.75% per day. Alternatively, the average loss of volume can be from 0.25% to 0.5% per day.

In a further embodiment, the present disclosure provides a composition wherein the compound is present in an amount of at least about 10% (w/w) of the coating such as in a mixture of the compound and the polymer. In another embodiment, the compound is present in an amount of at least about 20, 25, 30, 40, 50, 55, 60, 70, 75, 80 or 90% (w/w). In other embodiments, the compound is present in an amount of at least about 25% (w/w). In some other embodiments, the compound is present in an amount of at least about 50% (w/w). In still other embodiments, the compound is present in an amount of at least about 75% (w/w). One of skill in the art will appreciate that other compositions are useful in the present disclosure.

In another embodiment, the compounds of the present disclosure can be applied onto a stent without a polymer. In another embodiment, compounds of the present disclosure can be applied onto a stent as a coating containing a matrix of the compound and polymer. In another embodiment, the compound of the present disclosure can be applied on a stent with in a rate limiting barrier. The compounds of the present disclosure in the coating can be in an amorphous form. In other embodiment the compound in the coating can be fully or partially crystalline form. The polymer can be non degradable, partially degradable or fully degradable. The coating can also be a non-polymeric such as metallic coating. In another embodiment, the stent includes an underlayer coating disposed between the stent surface and the compounds of the present disclosure or compounds of the present disclosure-polymer matrix. Suitable underlayer coatings can be polymeric such as parylene C, parylene N, ethylene vinyl alcohol (EVOH), polycaprolactone, hydroxylated ethylvinyl acetate (EVA), or others or combination thereof or non polymeric such as metallic or ceramic or others.

The coatings can be applied by any of the different methods which include but are not limited to spraying, ultrasonic deposition, dipping, inkjet dispension, plasma deposition, ion implantation, sputtering, evaporation, vapor deposition, pyrolysis, electroplating, glow discharge coating, or others or combination thereof.

The total thickness of one or more coatings or layers, or the thickness per coating or layer of one or more coatings or layers, can range from about 1 nanometer to about 100 micrometers, or from about 100 nanometers to about 50 micrometers, or from about 1 micrometer to about 20 micrometers, or from about 1 micrometer to about 10 micrometers, or from about 0.5 micrometer or 1 micrometer to about 5 micrometers, or from about 0.5 micrometer or 1 micrometer to about 3 micrometers.

The compounds of the present disclosure can be mixed with an antioxidant or stabilizer to prevent degradation due to oxidation or other means. Antioxidants include but are not limited to butylated hydroxytoluene (BHT), ferrous sulfate, ascorbic acid and derivatives thereof (e.g., ascorbyl palmitate), ethylenediamine-tetra-acetic acid (EDTA), or others. Stabilizers include, but are not limited to, amglene, hydroquinone, quinine, sodium metabisulfite or others. Antioxidants and stabilizers can be combined with the compounds directly or blended with the compound formulation such as compound-polymer matrix to reduce conformation change or degradation during manufacturing processes and increase shelf life or storage life of the compounds or compound containing implant. The amount of an antioxidant, such as BHT, mixed with a compound of the disclosure can range from about 0.01% to about 10%, or from about 0.05% to about 5%, or from about 0.1% to about 3%. The amount of a stabilizer, such as amylene, mixed with a compound of the disclosure can range from about 0.01% to about 10%, or from about 0.05% to about 5%, or from about 0.1% to about 1%. One of skill in the art will appreciate that other antioxidants and stabilizers are useful in the present disclosure.

The compounds of the present disclosure can be administered in combination with an additional therapeutic agent such as anti-platelet, anti-thrombotic, anti-inflammatory, anti-angiogenic, anti-proliferative, immunosuppressant, anti-cancer or other agents or combinations thereof. One of skill in the art will appreciate that other therapeutic agents are useful in the present disclosure.

The additional therapeutic agents can be incorporated in or on the stent together with the compounds of the present disclosure and/or separately from compounds of the present disclosure. In one embodiment, the compound of the present disclosure and the additional therapeutic agent are matrixed together with a polymer and coated on an implant. In other embodiment, the compound and the additional therapeutic agent can be coated on at least a portion of the implant.

At least a portion of the additional therapeutic agent can be released from the stent prior to, concurrently or subsequent to the release of the compounds of the present disclosure from the implant. The additional therapeutic agent can also be administered separately through systemic or site-specific administration prior to, during or post delivery of compounds of the present disclosure.

For example, compounds of the present disclosure are administered with anti platelets or anti-thrombotics such as heparin, clopidogrel, coumadin, aspirin, ticlid or others. In another example, compounds of the present disclosure are given with anti-inflammatory agents such as aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone (relafen), acetaminophen, and mixtures thereof; COX-2 inhibitors, such as nimesulide, NS-398, flosulid, L-745337, celecoxib, rofecoxib, SC-57666, DuP-697, parecoxib sodium, JTE-522, valdecoxib, SC-58125, etoricoxib, RS-57067, L-748780, L-761066, APHS, etodolac, meloxicam, S-2474, and mixtures thereof; glucocorticoids, such as hydrocortisone, cortisone, prednisone, prednisolone, methylprednisolone, meprednisone, triamcinolone, paramethasone, fluprednisolone, betamethasone, dexamethasone, fludrocortisone, desoxycorticosterone, valdecoxib, dichlofenac, 6-MNA, L-743, L-337, NS-398, SC58125, ketorolac, clobetazol or others or analogues of the above or combinations thereof. In another example the compounds of the present disclosure are given with an immunosuppressant such as cyclosporine A, tacrolimus or others or analogues of the above or combinations thereof.

In some embodiments, the compounds of the present disclosure are administered alone or in combination with at least one additional therapeutic agent for treatment of an ophthalmic condition or disorder. Any suitable therapeutic agent known to one of skill in the art can be combined with the compounds of the present disclosure for use in the treatment of ophthalmic conditions or diseases. Therapeutic agents that can be combined with the compounds of the present disclosure include, but are not limited to, lucentis, avastin, macugan, volociximab, olopatadine, mydriatcs, dexamethasone, pilocarpine, tropicamide, quinolone, galentamine, fluocinolone acetonide, triamcinolone acetonide, atropine, atropine sulfate, atropine hydrochloride, atropine methylbromide, atropine methylnitrate, atropine hyperduric, atropine N-oxide, phenylephrine, phenylephrine hydrochloride, hydroxyamphetamine, hydroxyamphetamine hydrobromide, hydroxyamphetamine hydrochloride, hydroxyamphetamine iodide, cyclopentolate, cyclopentolate hydrochloride, homatropine, homatropine hydrobromide, homatropine hydrochloride, homatropine methylbromide, scopolamine, scopolamine hydrobromide, scopolamine hydrochloride, scopolamine methylbromide, scopolamine methylnitrate, scopolamine N-oxide, tropicamide, tropicamide hydrobromide, tropicamide hydrochloride, valdecoxib, celecoxib, rofecoxib, dichlofenac, etodolac, meloxicam, nimesulfide, 6-MNA, L-743, L-337, NS-398, SC58125, ketorolac, clobetazol, pilocarpine, isopilocarpine, physostigmine, quaternary ammonium compounds including stearyl ammonium chloride and benzyl ammonium chloride, and mixtures, ionic salts, and combinations thereof.

Formulations of the compounds of the present disclosure for ophthalmic uses can include poly(methyl methacrylate), poly(2-hydroxyethyl methacrylate) polymers (PHEMA), poly(dimethyl siloxane), poly(ethylene glycol), poly(ethylene glycol)-block-polyamino acid, hyaluronic acid, collagen, polypeptides, polysaccharides or any polymers described herein. The polymers useful in such formulations can be of any size. In some embodiments, the polymers can have a molecular weight of between about 5 kilo Daltons (kD) and 8,000 kD. One of skill in the art will appreciate that polymers of other sizes are useful in the present disclosure.

In some embodiments, the compounds of the present disclosure can be administrated alone or as part of compound-polymer formulation, compound-solvent formulation or compound-carrier formulation. All formulations of the present disclosure may include active and inactive ingredients. Other active ingredients include, but are not limited to, anti-inflammatory agents, immunomodulating agent and anti-infective agents, antioxidants, antibody, antibiotics, anti-angiogenics, anti-vascular endothelial growth factor agent, antihistamines and lubricant. Inactive ingredients include, but are not limited to, carrier, solvent, inorganic materials, pH-adjustor, radio-opaque, radioactive, fluorescent, NMR contrast or other “reporter or indicator” materials. Examples of solvent in the compound-solvent formulation include, but are not limited to, water, saline, alcohol, and dimethyl sulfoxide. Examples of carrier in the compound-carrier formulation are glycerin, paraffin, beeswax, ethylene glycol, propylene glycol, polyethylene glycol, and macrogels. Examples of inorganic materials include, but are not limited to, boric acid, calcium chloride, magnesium chloride, potassium chloride, sodium chloride, zinc chloride, sodium borate, povidone, and dibasic sodium phosphate. Examples of pH-adjustors include, but are not limited to, sodium hydroxide, hydrogen chloride, buffer, and other inorganic and organic acids and bases. Examples of preservatives include, but are not limited to, benzalkonium chloride, and a polyquaternium. Examples of lubricants include, but are not limited to, carboxymethylcellulose sodium, polyethylene glycol, propylene glycol and ethylene glycol. One of skill in the art will appreciate that other active and inactive ingredients, as well as solvents and carriers are useful in the present disclosure.

In other embodiments, the present disclosure provides a composition in a dosage form for systemic or local administration, which provides a total dose, or dose per administration or dose over a certain period (e.g., daily, weekly, biweekly, monthly, etc.), of a compound of the disclosure of from about 0.1 mg to about 20 mg. In some other embodiments, the total dose, or dose per administration or dose over a certain period, of the compound is from about 0.5 mg to about 10 mg, or from about 1 mg to about 5 mg, or from about 1 μg to about 1 mg, or from about 100 μg to about 1 mg, or from about 40 μg or 50 μg to about 500 μg, or from about 20 μg to about 200 μg.

### IV. Treatment

The compounds of the present disclosure can be used to treat diseases in mammals alone or in combination with other agents, including conditions such as:
a) Treatment and prevention of acute or chronic organ or tissue transplant rejection, e.g. for the treatment of recipients of heart, lung, combined heart-lung, liver, kidney, pancreatic, skin or corneal transplants. They can also be used for the prevention of graft-versus-host disease, such as following bone marrow transplantation.
b) Treatment and prevention of transplant vasculopathies, e.g. atherosclerosis.
c) Treatment and prevention of cell proliferation and migration leading to vessel intimal thickening, blood vessel obstruction, obstructive vascular atherosclerosis, restenosis.
d) Treatment and prevention of autoimmune disease and of inflammatory conditions, such as arthritis (for example rheumatoid arthritis, arthritis chronica progrediente and arthritis deformans) and rheumatic diseases.
e) Treatment and prevention of asthma.
f) Treatment of multi-drug resistance conditions such as multidrug resistant cancer or multidrug resistant AIDS.
g) Treatment of proliferative disorders, e.g. tumors, cancer, hyperproliferative skin disorder such as psoriasis and the like.
h) Treatment of infections such as fungal, bacterial and viral.
i) Treatment or prevention of cellular proliferation in vascular shunts.
j) Treatment or prevention of ophthalmic conditions and diseases such as wet or dry age related macular degeneration, uveitis, diabetic retinopathy, macular edema, post-laser or cataract surgical complications.
k) Prevention of neo-vascularization.
l) Treatment or prevention of fibrosis of organs
m) Treatment or prevention of adhesions

In some embodiments, the present disclosure provides a method of inhibiting cell proliferation in a subject in need thereof by site specific administration of a therapeutically effective amount of a compound myolimus, or a derivative thereof to the subject.

In some other embodiments, the administration of the compound of the present disclosure is via oral administration, administration as a suppository, topical contact, parenteral, intravascular, intravenous, intraperitoneal, intramuscular, intralesional, intranasal, pulmonary, mucosal, transdermal, ophthalmic, subcutaneous administration or intrathecal administration.

In still other embodiments, the administration of the compound of the present disclosure is via delivery from a temporary device such as a catheter or an permanent device such as an implant. Implant can be permanent or can biodegrade in physiological environment over time. In another embodiment, the temporary device is selected from the group consisting of a catheter, porous balloon, non-porous balloon, and an expandable membrane. In still another embodiment, the implant is a luminal prosthesis. In yet other embodiments, the luminal prosthesis comprises an expandable scaffold. In another embodiment, the luminal prosthesis comprises a stent or a graft. In still other embodiments, the luminal prosthesis is a vascular stent.

In other embodiments, the implant is a wrap. In another embodiment, the wrap is a vascular wrap which covers at least some portion of vascularature. In yet another embodiment, the wrap is a organ wrap which covers at least some portion of the organ.

One means of measuring the effectiveness of the compounds of the present disclosure include measuring effective concentration (EC₅₀).

In some embodiments, the present disclosure provides a method wherein the effective total dose, or dose per administration or dose over a certain period (e.g., daily, weekly, biweekly, monthly, etc.), of a compound of the disclosure is from about 0.1 mg to about 20 mg. In some other embodiments, the effective total dose, or dose per administration or dose over a certain period, of the compound is from about 0.5 mg to about 10 mg, or from about 1 mg to about 5 mg, or from about 1 μg to about 1 mg, or from about 100 μg to about 1 mg, or from about 40 μg or 50 μg to about 500 μg, or from about 20 μg to about 200 μg.

Matrix metalloproteinases (MMP-9) play a key role in cellular migration and proliferation including conditions such as neointimal growth and vascular remodeling after stent implantation. Release of MMPs cause increases in proteoglycan rich, extracellular matrix which increases smooth muscle cell migration after vascular injury. Plasma active MMP-9 levels may be a useful independent predictor of bare metal stent in stent restenosis. (Elevated Plasma Active Matrix Metalloproteinase-9 Level Is Associated With Coronary Artery In-Stent Restenosis, Arterioscler Thromb Vasc Biol. 2006; 26:e121-e125.). Compounds inhibiting production of MMP-9 can have therapeutic impact on treatment and prevention of inflammatory, proliferative and other disease conditions discussed above. Compounds of the present disclosure inhibit MMP-9 production as shown in Example 10.

In some embodiments, the compounds of the present disclosure provide greater inhibition of MMP-9 as compared to rapamycin.

Monocyte chemoattractant protein 1 (MCP-1) is a potent monocyte chemoattractant secreted by many cells in vitro, including vascular smooth muscle and endothelial cells. Eliminating MCP-1 gene or blockade of MCP-1 signals has been shown to decrease atherogenesis in hypercholesterolemic mice. MCP-1 has been shown to play a role in pathogenesis of neointimal hyperplasia in monkeys. (Importance of Monocyte Chemoattractant Protein-1 Pathway in Neointimal Hyperplasia After Periarterial Injury in Mice and Monkeys, Circ Res. 2002; 90:1167-1172.) MCP-1 is also strongly expressed in a small subset of cells in macrophage-rich regions of human and rabbit atherosclerotic lesions (Expression of Monocyte Chemoattractant Protein 1 in Macrophage-Rich Areas of Human and Rabbit Atherosclerotic Lesions, PNAS, Vol 88, 5252-5256). Compounds inhibiting production of MCP-1 can have therapeutic impact on treatment and prevention of inflammatory, proliferative and other disease conditions discussed above. Compounds of the present disclosure inhibit MCP-1 production as shown in Example 10.

In some other embodiments, the compounds of the present disclosure provide greater inhibition of MCP-1 as compared to rapamycin.

A. Ophthalmic Conditions and Diseases

In some embodiments, the compounds, pharmaceutical compositions and devices of the present disclosure are useful for the treatment of ophthalmic conditions and diseases. The compounds, pharmaceutical compositions and devices of the present disclosure are useful in the treatment of many ophthalmic condition or disease. Ophthalmic conditions and diseases that can be treated by the compounds and devices of the present disclosure include, but are not limited to, disorders of the eyelid, disorders of the lacrimal system and orbit, tear duct blockage, disorders of conjunctiva, disorders of the sclera, cornea, iris and ciliary body, disorders of the lens, disorders of the choroid and retina, Age-related Macular Degeneration (AMD), Diabetic Macular Edema (DME), glaucoma, uveitis, disorders of the vitreous body and globe, disorders of the optic nerve and visual pathways, disorders of the ocular muscles, binocular movement, accommodation and refraction, visual disturbances and blindness, etc. Additional ophthalmic conditions and diseases that can be treated with the compounds and devices of the present disclosure include inhibition of cell proliferation, prevention of inflammation, prevention of neovascularization, protection of neurovascular system, and prevention of immune response after transplantation. One of skill in the art will appreciate that other ophthalmic conditions and diseases can be treated using the compounds and devices of the present disclosure.

Current treatment methods include surgery and medications. Surgical treatment methods include retinal implant, high speed laser eye surgery, endothelial keratoplasty, cataract surgery, glaucoma surgery, refractive surgery, corneal surgery, vitreo-retinal surgery, eye muscle surgery, oculoplastic surgery, uses of stem cells to create corneas or part of corneas that can be transplanted into the eyes. The compounds of the present disclosure can be administered prior to, concurrent with or post any of the above procedures, alone or in conjunction with other therapeutic agents.

Ophthalmic conditions and diseases can be treated using compounds, pharmaceutical compositions and devices of the present disclosure, as described above. The compounds and pharmaceutical compositions of the present disclosure can be administered via any method known to one of skill in the art. In some embodiments, the compounds of the present disclosure are administered via implant, injection or eye drop. In some other embodiments, the administration is through an intraocular or intravitreal body of the eye. In one embodiment, the administration is via an implant. In other embodiments, the compounds are administered via an implant where the compound is released via a metallic, ceramic or polymer coating.

When administration is via an implant, the compound can be released by any means described herein. In some embodiments, release of the compound from the implant can be via osmotic pressure or diffusion.

In some embodiments, the compounds of the present disclosure are combined with at least one other therapeutic agent for treatment of an ophthalmic condition or disorder. Any suitable therapeutic agent known to one of skill in the art can be combined with the compounds of the present disclosure for use in the treatment of ophthalmic conditions or diseases. In some embodiments, the additional therapeutic agents include, but are not limited to, anti-inflammatory agents, immunomodulating agents, anti-infective agents, antioxidants, antibodies, antibiotics, anti-angiogenics, anti-vascular endothelial growth factor agents, antihistamines and lubricants. Therapeutic agents that can be combined with the compounds of the present disclosure include, but are not limited to, lucentis, avastin, macugan, volociximab, olopatadine, mydriatcs, dexamethasone, pilocarpine, tropicamide, quinolone, galentamine, fluocinolone acetonide, triamcinolone acetonide, atropine, atropine sulfate, atropine hydrochloride, atropine methylbromide, atropine metliylnitrate, atropine hyperduric, atropine N-oxide, phenylephrine, phenylephrine hydrochloride, hydroxyamphetamine, hydroxyamphetamine hydrobromide, hydroxyamphetamine hydrochloride, hydroxyamphetamine iodide, cyclopentolate, cyclopentolate hydrochloride, homatropine, homatropine hydrobromide, homatropine hydrochloride, homatropine methylbromide, scopolamine, scopolamine hydrobromide, scopolamine hydrochloride, scopolamine methylbromide, scopolamine methylnitrate, scopolamine N-oxide, tropicamide, tropicamide hydrobromide, tropicamide hydrochloride, pilocarpine, isopilocarpine, valdecoxib, celecoxib, rofecoxib, dichlofenac, etodolac, meloxicam, nimesulfide, 6-MNA, L-743, L-337, NS-398, SC58125, ketorolac, clobetazol, physostigmine, quaternary ammonium compounds including stearyl ammonium chloride and benzyl ammonium chloride, and mixtures, ionic salts, and combinations thereof.

It can be appreciated that all embodiments disclosed in the present disclosure can be utilized alone or in combination with any other embodiments or examples in this disclosure.

### V. Examples

### Example 1

### Biological Activity of Myolimus

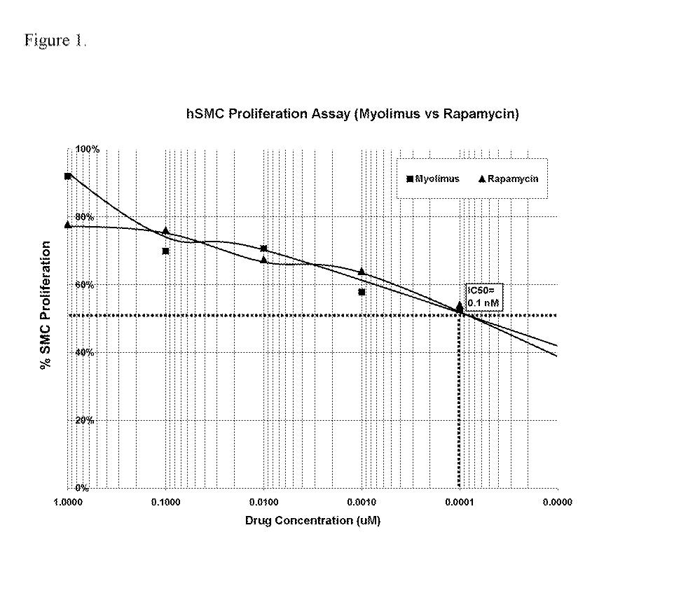

Potency of myolimus was demonstrated by in vitro human smooth muscle cell culture testing. The amounts of incorporated thymidine for samples of myolimus of varying concentrations (0.0001, 0.001, 0.01, 0.1, and 1 μM) and of rapamycin of varying concentrations (0.0001, 0.001, 0.01, 0.1, and 1 μM) were measured after exposure for 8 hours (as shown in FIG. 1). The IC₅₀ of both myolimus and rapamycin was 0.1 nM indicating potent anti-proliferative properties.

### Example 2

### Preparation of Myolimus Eluting Stent with Durable Polymer

15 mg poly(n-butyl methacrylate) (PBMA) was dissolved into 3 mL dichloromethane at room temperature. 10 mg of myolimus was placed in a vial and dissolved in 2 mL dichloromethane with or without 0.1% (w/w) BHT. The solutions were combined and further diluted with 10 mL dichloromethane.

The stent was loaded on a wire mandrel and rotated at 200 rpm and a micro-blaster with a 0.020″ (0.5 nm) diameter nozzle was turned yo provide micro-blasting with a 20 μm diameter media. The nozzle traverses along the stent axially at a rate of 2 seconds per inch back and forth for a total of 5 cycles. The stent direction is reversed and micro-blasting is repeated. The stent is then precrimped to a smaller inner diameter such as 0.036″ (0.91 nm). It can be appreciated that the parameters used for surface texturing may vary.

A microprocessor-controlled ultrasonic sprayer was used to apply approximately 100 ug (2.2 ug myolimus/mm stent) of the drug containing PBMA solution to the entire surface of a 18 mm metal stent (available from Elixir Medical Corp, Sunnyvale, Calif.). After coating, the stent was placed in a vacuum chamber. The stent was then mounted on the balloon of a 3.0×20 mm PTCA delivery catheter. The catheter was then inserted in a coil and packaged in a Tyvek® pouch. The pouch was sterilized by ethylene oxide. The Tyvek® pouch was further packaged in a foil pouch with oxygen scavengers and nitrogen purge and vacuum sealed.

### Example 3

### Preparation of Myolimus Eluting Stents with Bioabsorbable Polymer

5 mg poly(ethylene carbonate) was dissolved into 1 mL dichloromethane at room temperature. 10 mg of myolimus was placed in a vial and dissolved in 2 mL dichloromethane with or without 0.1% (w/w) BHT. The solutions were combined and further diluted with 6 mL dichloromethane.

A microprocessor-controlled ultrasonic sprayer was used to apply around 125 ug (2.7 ug myolimus/mm stent) of the drug containing PEC solution to the entire surface of a 18 mm metal stent (available from Elixir Medical Corp, Sunnyvale, Calif.). After coating, the stent was placed in a vacuum chamber. The stent was then mounted on the balloon of a 3.0×20 mm PTCA delivery catheter. The catheter was then inserted in a coil and packaged in a Tyvek® pouch. The pouch was sterilized by ethylene oxide. The Tyvek® pouch was further packaged in a foil pouch with oxygen scavengers and nitrogen purge and vacuum sealed.

### Example 4

### In vivo Testing of Myolimus Eluting Stent with Durable Polymer

The efficacy of a myolimus eluting stent system with durable polymer (as prepared above from Example 2) for 40 ug drug loading (2.2 ug/mm stent) and 120 ug drug loading (6.7 ug/mm stent) was evaluated at 28±2 day and 90±2 day angiographic outcomes respectively in porcine coronary arteries in the non-diseased porcine coronary artery model. The control stents were Cypher™ Sirolimus (rapamycin) eluting Coronary Stent (Cordis Corporation).

The nonatherosclerotic swine model was chosen as this model has been used extensively for stent and angioplasty studies resulting in a large volume of data on the vascular response properties and its correlation to human vascular response (Schwartz et al, Circulation. 2002; 106:1867-1873). The animals were housed and cared for in accordance the Guide for the Care and Use of Laboratory Animals as established by the National Research Council.

All animals were pretreated with aspirin (325 mg) and clopidogel (75 mg) per oral dose beginning at least 3 days prior to the intervention and continuing for the duration of the study. After induction of anesthesia, the left or right femoral artery was accessed using standard techniques and an arterial sheath was introduced and advanced into the artery.

Vessel angiography was performed under fluoroscopic guidance, a 7 Fr. guide catheter was inserted through the sheath and advanced to the appropriate location where intracoronary nitroglycerin was administered. A segment of coronary artery ranging from 2.25 to 4.0 mm mean lumen diameter was chosen and a 0.014″ guidewire inserted. Quantitative Coronary Angiography (QCA) was performed to document the reference vessel diameter.

The appropriately sized stent was advanced to the deployment site. The balloon was inflated at a steady rate to a pressure sufficient to achieve a balloon to artery ratio of 1.30:1.0 for 28 days and 1.1:1 (low injury) for 90 days, respectively. Pressure was maintained for approximately 10 seconds. Angiography was performed to document post-procedural vessel patency and diameter.

Follow-up angiography was performed at the designated endpoint for each of the animals. Each angiogram was qualitatively evaluated for evidence of stent migration, lumen narrowing, stent apposition, presence of dissection or aneurysms, and flow characteristics. Upon completion of follow-up angiography, the animals were euthanized.

The hearts were harvested from each animal and the coronary arteries were perfused with 10% buffered formalin at 100 to 120 mm Hg. The hearts were immersed in 10% buffered formalin. Any myocardial lesions or unusual observations were reported.

Angiographic parameters measured or calculated included:
Marginal vessel (proximal and distal) mean lumen diameter (post-stent and final only)
Mean lumen diameter of the target region (all angiograms)
Minimal lumen diameter (MLD) of the target region (post-stent and final only)
Diameter stenosis [1−(MLD/RVD)]×100% where RVD is a calculation of the reference diameter at the position of the obstruction (measure obtained by a software-based iterative linear regression technique to generate an intrapolation of a projected vessel without the lesion) (final angiogram only)
Balloon to artery ratio [balloon/pre-stent mean luminal diameter]
Stent to artery ratio [post-stent/pre-stent mean luminal diameter]
Late loss ratio [MLD final-MLD post-stent]

All animal survived to the designated end point. There were no documented incidents of stent migration, stent malapposition, persistent dissection or evidence of aneurysm. Three outlying data points (total occlusion or near total occlusion) for the Cypher Stent were excluded. The average percent stenosis at 28 days for the myolimus stent with durable polymer with 40 ug dose (approx. 2.2 microgram/mm length drug dose) was 38±11 (n=15) as compared to Cypher Stent 170 ug dose (approx. 9.5 microgram/mm length drug dose) pooled data from this and previous studies with similar protocols which provided an average percent stenosis of 36±14 (n=37) for Cypher stents.

The average percent stenosis at 90 days for the myolimus stent with durable polymer with 120 ug dose (approx. 6.7 microgram/mm length drug dose) was 40±21 (n=7) as compared to Cypher Stent pooled data from this and previous studies with similar protocols which provided an average percent stenosis of 53±21 (n=16) for Cypher stents.

The myolimus eluting stents with durable polymer with a drug dose of approx. 2.2 microgram/mm length drug dose in this example when implanted in the porcine model at 28 days resulted in similar percentage stenosis as compared to that of the Cypher Stent which has a significantly higher dose of approx. 9.5 microgram/mm length drug dose. The myolimus eluting stents with durable polymer with the approx 6.7 microgram/mm length drug dose in this example when implanted in the porcine model at 90 days resulted in lower percentage stenosis as compared to that of the Cypher Stent.

### Example 5

### In Vivo Testing of Myolimus Eluting Stents with Bioabsorbable Polymer

The efficacy of a myolimus eluting stent with bioabsorbable polymer (as prepared above from Example 3) with approx. 2.5 microgram/mm length drug dose was evaluated by comparing 28±2 day and 90±2 angiographic outcomes in porcine coronary arteries to the rapamycin eluting stent system, Cypher™ Coronary Stent (Cordis Corporation) in the non-diseased porcine coronary artery model.

The nonatherosclerotic swine model was chosen as this model has been used extensively for stent and angioplasty studies resulting in a large volume of data on the vascular response properties and its correlation to human vascular response (Schwartz et al, Circulation. 2002; 106:1867-1873). The animals were housed and cared for in accordance the Guide for the Care and Use of Laboratory Animals as established by the National Research Council.

All animals were pretreated with aspirin (325 mg) and clopidogel (75 mg) per oral dose beginning at least 3 days prior to the intervention and continuing for the duration of the study. After induction of anesthesia, the left or right femoral artery was accessed using standard techniques and an arterial sheath was introduced and advanced into the artery.

Vessel angiography was performed under fluoroscopic guidance, a 7 Fr. guide catheter was inserted through the sheath and advanced to the appropriate location where intracoronary nitroglycerin was administered. A segment of coronary artery ranging from 2.25 to 4.0 mm mean lumen diameter was chosen and a 0.014″ guidewire inserted. Quantitative Coronary Angiography (QCA) was performed to document the reference vessel diameter.

The appropriately sized stent was advanced to the deployment site. The balloon was inflated at a steady rate to a pressure sufficient to achieve a balloon to artery ratio of 1.30:1.0 for 28 days and 1.1:1 (low injury) for 90 days, respectively. Pressure was maintained for approximately 10 seconds. Angiography was performed to document post-procedural vessel patency and diameter.

Follow-up angiography was performed at the designated endpoint for each of the animals. Each angiogram was qualitatively evaluated for evidence of stent migration, lumen narrowing, stent apposition, presence of dissection or aneurysms, and flow characteristics. Upon completion of follow-up angiography, the animals were euthanized.

The hearts were harvested from each animal and the coronary arteries were perfused with 10% buffered formalin at 100 to 120 mm Hg. The hearts were immersed in 10% buffered formalin. Any myocardial lesions or unusual observations were reported.

Angiographic parameters measured or calculated included:
Marginal vessel (proximal and distal) mean lumen diameter (post-stent and final only)
Mean lumen diameter of the target region (all angiograms)
Minimal lumen diameter (MLD) of the target region (post-stent and final only)
Diameter stenosis [1−(MLD/RVD)]×100% where RVD is a calculation of the reference diameter at the position of the obstruction (measure obtained by a software-based iterative linear regression technique to generate an intrapolation of a projected vessel without the lesion) (final angiogram only)
Balloon to artery ratio [balloon/pre-stent mean luminal diameter]
Stent to artery ratio [post-stent/pre-stent mean luminal diameter]
Late loss ratio [MLD final-MLD post-stent]

All animal survived to the designated end point. There were no documented incidents of stent migration, stent malapposition, persistent dissection or evidence of aneurysm. Three outlying data points (total occlusion or near total occlusion) for the Cypher Stent were excluded. The average percent stenosis at 28 days for the myolimus eluting stent with bioabsorbable polymer (approx. 2.5 microgram/mm length drug dose) was 35±17 (n=15) as compared to Cypher Stent (approx. 9.5 microgram/mm length drug dose) pooled data from this and previous studies with similar protocols which provided an average percent stenosis of 36±14 (n=37) for Cypher stents. The average percent stenosis at 90 days for the myolimus eluting stent with bioabsorbable polymer (approx. 2.5 microgram/mm length drug dose) was 33±9 (n=6) as compared to Cypher Stent pooled data from this and previous studies with similar protocols which provided an average percent stenosis of 53±21 (n=16) for Cypher stents.

The myolimus eluting stent with bioabsorbable polymer with approx. 2.5 microgram/mm length drug dose in this example when implanted in the porcine model at 28 days resulted in similar percentage stenosis as compared to that of the Cypher Stent which has a significantly higher dose of approx. 9.5 microgram/mm length drug dose. Myolimus eluting stent with bioabsorbable polymer with approx. 2.8 microgram/mm length drug dose in this example when implanted in the porcine model at 90 days resulted in lower percentage stenosis as compared to that of the Cypher Stent with significantly higher dose of approx. 9.5 microgram/mm length drug dose.

### Example 6

### In Vivo Pharmacokinetics of Myolimus Eluting Stents with Durable Polymer

Pharmacokinetic evaluation of the myolimus stent with durable polymer system from Example 2 was performed at 6 hours, 3 days, 7 days, 28 days, 90 days and 180 days in the porcine coronary artery model. The interventional procedures used were similar to the in vivo angiographic study described in Example 4 up to stent implantation.

The appropriately sized stent was advanced to the deployment site. The balloon was inflated at a steady rate to a pressure sufficient to achieve a balloon to artery ratio of 1:1. Pressure was maintained for approximately 10 seconds. Angiography was performed to document post-procedural vessel patency and diameter. A total of 9 stents (3 per time point) were implanted.

At the appropriate time point the animals were euthanized and the hearts excised. The stented segment including approximately 10 mm of vessel proximal and 10 mm distal to the stented section was excised. The proximal and distal sections were separated and stored in separate vials. The tissue surrounding the stent was carefully removed from stent and each place in separate vials. All were then frozen to −70° C. prior to being analyzed using liquid chromatography mass spectroscopy (LCMS).

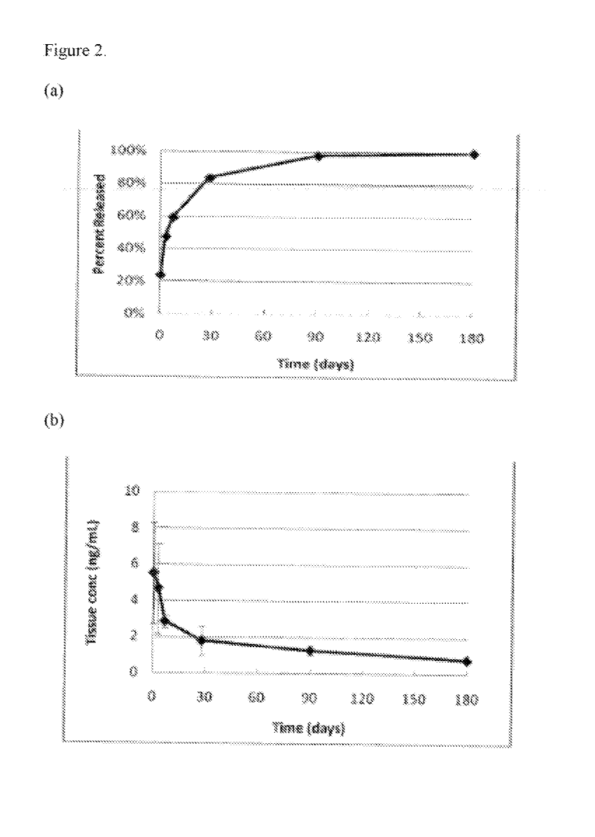

All animal survived to the designated end point. The myolimus eluting stent, in this example demonstrates release of myolimus from the stent with greater than 80% of the drug released at 28 days, greater than 95% of the drug released at 90 days and almost completely released from the stent at 180 days (FIG. 2 a).

The myolimus eluting stent in this example demonstrates that the average myolimus tissue concentration at 28 days is approximately 2 ng drug per mg tissue, at 90 days is close to 1 ng per mg of tissue, and at 180 days is less than 0.5 ng drug per mg tissue (FIG. 2b).

### Example 7

### In Vivo Pharmacokinetics of Myolimus Eluting Stents with Bioabsorbable Polymer

Pharmacokinetic evaluation of the myolimus eluting stent with bioabsorable polymer system from Example 3 was performed at 6 hours, 3 days, 7 days, and 28 days in the porcine coronary artery model. The interventional procedures used were similar to the in vivo angiographic study described in Example 4 up to stent implantation.

The appropriately sized stent was advanced to the deployment site. The balloon was inflated at a steady rate to a pressure sufficient to achieve a balloon to artery ratio of 1:1. Pressure was maintained for approximately 10 seconds. Angiography was performed to document post-procedural vessel patency and diameter. A total of 9 stents (3 per time point) were implanted.

At the appropriate time point the animals were euthanized and the hearts excised. The stented segment including approximately 10 mm of vessel proximal and 10 mm distal to the stented section was excised. The proximal and distal sections were separated and stored in separate vials. The tissue surrounding the stent was carefully removed from stent and each place in separate vials. All were then frozen to −70° C. prior to being analyzed using liquid chromatography mass spectroscopy (LCMS).

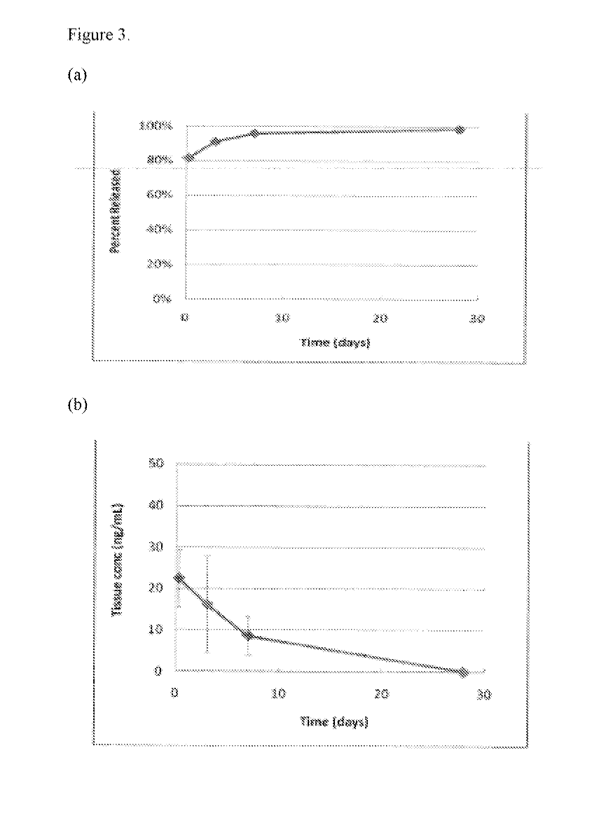

All animal survived to the designated end point. The myolimus eluting stent in this example demonstrates release of myolimus from the stent with greater than 95% of the drug released at 28 days (FIG. 3(a)).

The myolimus eluting stent in this example demonstrates that the average myolimus tissue concentration at 28 days is approximately 0.1 ng drug per mg tissue (FIG. 3(b)).

### Example 8

### Preparation and In Vivo Testing of Myolimus Eluting Stents without Polymer Coating

10 mg of myolimus was placed in a vial and dissolved in 6 mL dichloromethane with or without 0.1% (w/w) BHT. A microprocessor-controlled ultrasonic sprayer was used to apply around 20 ug (1.1 ug myolimus/mm stent) of the drug on to the entire surface of a 18 mm metal stent (available from Elixir Medical Corp, Sunnyvale, Calif.). After coating, the stent was placed in a vacuum chamber. The stent was then mounted on the balloon of a 3.0×20 mm PTCA delivery catheter. The catheter was then inserted in a coil and packaged in a Tyvek® pouch. The pouch was sterilized by ethylene oxide. The Tyvek® pouch was further packaged in a foil pouch with oxygen scavengers and nitrogen purge and vacuum sealed.

The efficacy of a myolimus eluting stent system with no polymer coating or BHT for approx. 1.1 microgram/mm length drug dose (20 ug drug loading) was evaluated by comparing 28±2 day histological outcomes in porcine coronary arteries to the myolimus eluting stent with 2.2 microgm/mm length drug dose (40 ug drug loading) of Example 4 in the non-diseased porcine coronary artery model with the balloon inflated at a steady rate to a pressure sufficient to achieve a balloon to artery ratio of 1.30:1.0. The % area stenosis was found to be 41% for both the myolimus eluting stent system with durable coating and myolimus eluting stent system with no polymer coating group after 28 days.

Myolimus eluting stent with no polymer coating with a significantly low dose of 1.1 microgram/mm length drug dose in this animal study is similar in efficacy as compared to Cypher Stent (approx. 9.5 microgram/mm length drug dose) pooled data from previous studies with similar protocols which provided an average percent stenosis of 36±14 (n=37) for Cypher stents.

### Example 9

### Preparation of Myolimus Eluting Balloons with or without Bioabsorable Polymer

5 mg poly(ethylene-carbonate) (PEC) was dissolved into 1 mL dichloromethane at room temperature. 10 mg of myolimus was placed in a vial and dissolved in 2 mL dichloromethane with or without 0.1% (w/w) BHT. The solutions were combined and further diluted with 6 mL dichloromethane.

A microprocessor-controlled ultrasonic sprayer was used to apply around 150 ug (5.6 ug myolimus/mm balloon) of the drug containing PEC solution to the folded balloon of a 3.0×20 mm PTCA catheter between the gold markers while the balloon was rotated. After coating, the catheter was placed in a vacuum chamber. The catheter was then inserted in a coil and packaged in a Tyvek® pouch. The pouch was sterilized by ethylene oxide. The Tyvek® pouch was further packaged in a foil pouch with oxygen scavengers and nitrogen purge and vacuum sealed.

The balloon is removed from the coil and tracked into the artery with blood at physiologic temperature of 37° C. Due to the low glass transition temperature (Tg) of the PEC (approx 20° C.), the polymer softens and becomes tacky. When the balloon is expanded, the drug-polymer matrix sticks to the surface of the artery delivering drug to the target tissue.

In another example, myolimus was mixed in dichloromethane without PEC and the resulting mixture was then sprayed on the balloon.

Optionally, a anti-inflammatory therapeutic agent, dexamethasone (5 mg), was mixed with the 10 mg myolimus and was placed in a vial and dissolved in 2 mL dichloromethane and sprayed on the balloon.

### Example 10

### Cytokine Inhibition by Macrocyclic Lactone

In cell culture studies, macrophages were activated to secrete cytokines such as MMP-9 and MCP-1 by treating the cells to E Coli lippopolysaccharide (LPS). Inhibition of these cytokines upon treatment of the activated macrophages with myolimus and rapamycin with 10 nM concentration was tested using ELISA assay.

MMP-9 levels in the 1st, 3rd and 7th day after stent implantation were positively correlated to the late loss index 6 months after stent implantation (Elevated matrix metalloproteinase expression after stent implantation is associated with restenosis. Int J. Cardiol. 2006; 112(1):85-90).

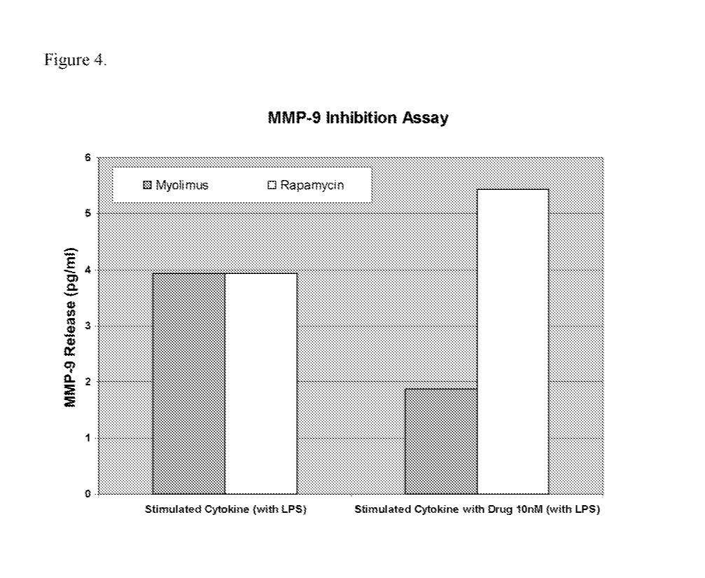

Myolimus reduced the production of the cytokine MMP-9 as compared to Rapamycin. Rapamycin in this study increased the production of MMP-9 (FIG. 4).

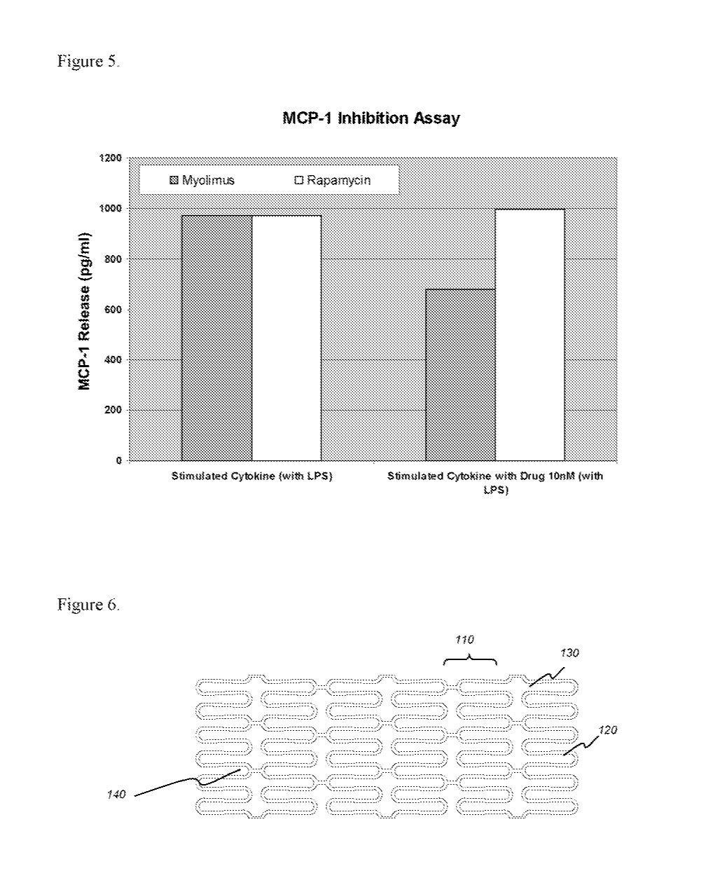

Myolimus reduced the production of cytokine MCP-1 as compared to rapamycin which did not have any impact on production of MCP-1 (FIG. 5).

Compounds used in drug delivery system in the present disclosure, such as myolimus, can provide better therapeutic response and higher levels of anti-cell proliferative and anti-cell migratory effect by providing greater inhibition of pro-proliferative and migration cytokines such as MCP-1 and MMP-9.

### Example 11

### Testing of Myolimus Eluting Stents with Durable Polymer in Human Clinical Trial

Clinical testing of the myolimus coated stent with durable polymer was conducted on 15 human subjects. Safety of the myolimus coated stent with durable polymer was evaluated clinically through the evaluation of major adverse cardiac events defined as: death, myocardial infarction (both Q-wave and non-Q-wave), and target lesion revascularization. Efficacy was evaluated through angiographic and intravascular ultrasound (IVUS) results at 6 months. The primary endpoint of the study was angiographic in-stent late lumen loss. Secondary endpoints were Major Adverse Cardiac Events (MACE) and additional angiographic and IVUS evaluation. The clinical study was approved by local Ethics Committee and all patients signed an Ethics approved informed consent before entry into the clinical study.

All patients were pretreated with aspirin and clopidogrel (300 mg) per oral beginning at least 1 day prior or on the day of the index procedure. Aspirin (>100 mg/day and Clopidogrel (75 mg/day) was continued through twelve months. In accordance with hospital standard percutaneous practice, the left or right femoral artery was accessed using standard techniques and an arterial sheath was introduced and advanced into the artery.

Index procedure vessel angiography was performed under fluoroscopic guidance, a 6 or 7 Fr. guide catheter was inserted through the sheath and advanced to the appropriate location; intracoronary nitroglycerin was administered per protocol. A segment of coronary artery ranging from 3.0 mm to 3.5 mm mean lumen diameter was chosen and a 0.014″ guidewire inserted. Quantitative Coronary Angiography (QCA) was performed to document the reference vessel diameter. Predilatation of the lesion was performed prior to stent implantation using standard technique.

Following predilatation, the appropriately sized stent (3.0×18 mm or 3.5×18 mm was advanced to the target lesion. The balloon was inflated at a steady rate to a pressure to fully deploy the stent. Pressure was maintained for approximately 30 seconds. Post dilatation of the stent could be performed as needed to assure good stent apposition to the vessel wall. Angiographic and intravascular ultrasound imaging (IVUS) was performed and recorded.

Follow-up angiography and IVUS was performed at the designated endpoint of 6 months. Each angiogram and IVUS image was qualitatively evaluated for evidence of lumen narrowing, stent apposition, and flow characteristics.

Angiographic and IVUS parameters measured or calculated included:
Marginal vessel (proximal and distal) mean lumen diameter (post-stent and final)
Mean lumen diameter of the target region (all angiograms)
Minimal lumen diameter (MLD) of the target region (post-stent and final only)
Diameter stenosis [1−(MLD/RVD)]×100%] where RVD is a calculation of the reference diameter at the position of the obstruction (measure obtained by a software-based iterative linear regression technique to generate an intrapolation of a projected vessel without the lesion) (final angiogram only).
In-stent Late Lumen Loss [MLD final-MLD post-stent]
In-stent percent neointimal volume as assessed by IVUS

Patients underwent 6 month clinical and angiographic follow-up. No patients experience any major adverse cardiac events during the follow-up period. Angiographic results demonstrated that the primary endpoint of angiographic in-stent late lumen loss was 0.15±0.11 mm. IVUS analysis was conducted on 14 of 15 patients and the results demonstrated in-stent percent neointimal volume of 1.4±1.2%.

As a comparison, Cypher stent tested in a previous pilot study and demonstrated similar clinical safety with no clinical events and angiographic results at 6 months of in-stent late lumen loss for the slow release group (the current commercially available formulation) of 0.09±0.3 mm and in-stent percent neiontimal volume by IVUS to be 0.3±0.6% (Sousa, J E, Circulation 2001; 103; 192-195).

### Example 12

### Testing of Myolimus Eluting Stents with Bioabsorable Polymer in Human Clinical Trial

Clinical testing of the myolimus coated stent with bioerodable polymer was conducted on 30 human subjects. Safety of the myolimus coated stent with bioerodable polymer was evaluated clinically through the evaluation of major adverse cardiac events defined as: death, myocardial infarction (both Q-wave and non-Q-wave), and target lesion revascularization. Efficacy was evaluated through angiographic and intravascular ultrasound (IVUS) results at 6 months for first group of 15 patients and 9 months for the second group of 15 patients. The primary endpoint of the study was angiographic in-stent late lumen loss. Secondary endpoints were Major Adverse Cardiac Events (MACE) and additional angiographic and IVUS evaluation. The clinical study was approved by local Ethics Committee and all patients signed an Ethics approved informed consent before entry into the clinical study.

All patients were pretreated with aspirin and Clopidogrel (300 mg) per oral beginning at least 1 day prior or on the day of the index procedure. Aspirin (>100 mg/day and Clopidogrel (75 mg/day) were continued through for at least 12 months. In accordance with hospital standard percutaneous practice, the left or right femoral artery was accessed using standard techniques and an arterial sheath was introduced and advanced into the artery.

Index procedure vessel angiography was performed under fluoroscopic guidance, a 6 or 7 Fr. guide catheter was inserted through the sheath and advanced to the appropriate location; intracoronary nitroglycerin was administered. A segment of coronary artery ranging from 3.0 mm to 3.5 mm mean lumen diameter was chosen and a 0.014″ guidewire inserted. Quantitative Coronary Angiography (QCA) was performed to document the reference vessel diameter. Predilatation of the lesion was performed prior to stent implantation using standard technique.

Following predilatation, the appropriately sized stent (3.0×18 mm or 3.5×18 mm was advanced to the target lesion. The balloon was inflated at a steady rate to a pressure to fully deploy the stent. Pressure was maintained for approximately 30 seconds. Post dilatation of the stent could be performed as needed to assure good stent apposition to the vessel wall. Angiographic and intravascular ultrasound imaging (IVUS) was performed and recorded.

Follow-up angiography and IVUS was performed at the designated endpoint of 6 months for 14 of the 15 patients from group one. Each angiogram was qualitatively evaluated for evidence of lumen narrowing, stent apposition, and flow characteristics.

Angiographic and IVUS parameters measured or calculated included:
Marginal vessel (proximal and distal) mean lumen diameter (post-stent and final)
Mean lumen diameter of the target region (all angiograms)
Minimal lumen diameter (MLD) of the target region (post-stent and final only)
Diameter stenosis [1−(MLD/RVD)]×100%] where RVD is a calculation of the reference diameter at the position of the obstruction (measure obtained by a software-based iterative linear regression technique to generate an intrapolation of a projected vessel without the lesion) (final angiogram only).
In-stent Late Lumen Loss [MLD final-MLD post-stent]
In-stent percent neointimal volume as assessed by IVUS

With one patient withdrawing from the study, 14 of 15 patients underwent 6 month clinical and angiographic follow-up. No patients experience any major adverse cardiac events during the follow-up period. Angiographic results demonstrated that the primary endpoint of angiographic in-stent late lumen loss was 0.37±0.44 (n=14) mm. IVUS analysis was conducted on 14 of 15 patients and the results demonstrated in-stent percent neointimal volume of 14.2±7.7%.

As a comparison, Cypher stent tested in a pilot study and demonstrated similar clinical safety with no clinical events and angiographic results at 6 months of in-stent late lumen loss for the slow release group (the current commercially available formulation) of 0.09±0.3 mm and in-stent percent neiontimal volume by IVUS to be 0.3±0.6% (Sousa, J E, Circulation 2001; 103; 192-195).

Although the foregoing disclosure has been described in some detail by way of illustration and example for purposes of clarity of understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A medical device for intracorporeal use, the medical device comprising:
an implant or a temporary device; and
at least one source comprising a macrocyclic lactone of a compound of formula (I): 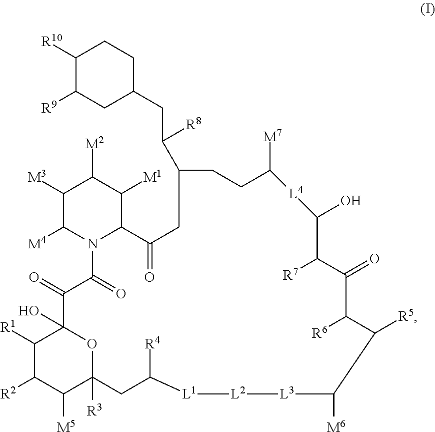
wherein:
R¹, R², R³, R⁵, R⁶, R⁸, M¹, M², M³, M⁴, M⁵, M⁶ and M⁷ are each independently H, —OH, C₁₋₆ alkyl or C₁₋₆ hydroxyalkyl;
R⁴, R⁷ and R⁹ are each independently H, —OH or C₁₋₆ alkoxy;
R¹⁰ is H, —OH, —OP(O)Me₂, 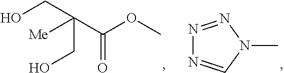
—O—(CH₂)ₙ—OH or —O—(CH₂)ₘ—O—(CH₂)ₒ—CH₃, wherein subscripts n and m are each independently from 2 to 8 and subscript o is from 1 to 6;
L¹ and L⁴ are each independently 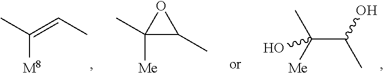
wherein each M⁸ is independently C₁₋₆ alkyl or C₁₋₆ hydroxyalkyl; and
L² and L³ are each independently 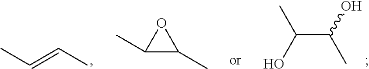
with the proviso that the macrocyclic lactone is not myolimus; and
wherein the amount of the macrocyclic lactone associated with the medical device is from about 10 micrograms/cm² to about 400 micrograms/cm².

2. The medical device of claim 1, wherein the medical device is configured to release the macrocyclic lactone to a lumen or organ of a mammalian body to inhibit cell proliferation, inflammation or cytokine production.

3. The medical device of claim 2, wherein the medical device is configured to release the macrocyclic lactone to a lumen or organ of a mammalian body to inhibit smooth muscle cell proliferation and inflammation.

4. The medical device of claim 1, wherein the implant is a luminal prosthesis.

5. The medical device of claim 4, wherein the luminal prosthesis comprises an expandable scaffold.

6. The medical device of claim 4, wherein the luminal prosthesis comprises a stent or a graft.

7. The medical device of claim 6, wherein the luminal prosthesis is a vascular stent.

8. The medical device of claim 7, wherein the stent is substantially fully degradable.

9. The medical device of claim 7, wherein the stent is balloon expandable.

10. The medical device of claim 4, wherein the luminal prosthesis has a luminal surface and a tissue-facing surface, and wherein the macrocyclic lactone is associated with at least one of the luminal and tissue-facing surfaces.

11. The medical device of claim 1, wherein at least 75% of the macrocyclic lactone is released from the medical device in a period from about 1 day to about 2 years.

12. The medical device of claim 1, wherein at least 90% of the macrocyclic lactone is released from the medical device in a period from about 1 day to about 6 months.

13. The medical device of claim 1, wherein at least 90% of the macrocyclic lactone is released from the medical device in a period from about 1 week to about 3 months.

14. The medical device of claim 1, wherein at least one source further comprises an additional therapeutic agent.

15. The medical device of claim 14, wherein the additional therapeutic agent is selected from the group consisting of anti-platelet agents, anti-thrombotic agents, anti-inflammatory agents, anti-angiogenic agents, anti-proliferative agents, immunosuppressants, and anti-cancer agents.

16. The medical device of claim 14, wherein the additional therapeutic agent is released prior to, concurrent with, or subsequent to release of the macrocyclic lactone.

17. The medical device of claim 14, wherein the macrocyclic lactone is released from a first source and the additional therapeutic agent is released from a second source.

18. The medical device of claim 14, wherein the macrocyclic lactone and the additional therapeutic agent are released from a single source.

19. The medical device of claim 1, wherein the source is contained in a polymeric material.

20. The medical device of claim 19, wherein the polymeric material is selected from the group consisting of polyurethanes, polyethylene imines, ethylene vinyl alcohol copolymers, silicones, C-flex, nylons, polyamides, polyimides, polytetrafluoroethylene (PTFE), parylene, parylast, poly(methacrylate), poly(vinyl chloride), poly(dimethyl siloxane), poly(ethylene vinyl acetate), polycarbonates, polyacrylamide gels, poly(methyl methacrylate), poly(n-butyl methacrylate), poly(butyl methacrylate) copolymer or blended with poly(ethylene vinyl acetate), poly(2-hydroxyethyl methacrylate), poly(ethylene glycol methacrylates), poly-styrene-b-isobutylene-b-styrene, copolymers of vinylidene fluoride and hexafluoropropylene, poly(ethylene carbonate), polylactides, poly(L-lactide), poly(L-lactide-glycolide) copolymers, poly(L-lactide-trimethylene carbonate) copolymers, poly(L-lactide-co-ε-caprolactone), salicylate based polyanhydride esters, salicylic acid-co-adipic acid-co-salicylic acid, salicylic acid-co-polylactide anhydride-salicylic acid, phosphoryl choline, and combinations thereof.

21. The medical device of claim 20, wherein the polymeric material is selected from the group consisting of poly(ethylene carbonate), poly(L-lactide-glycolide) copolymers, and poly(n-butylmethacrylate).

22. The medical device of claim 19, wherein the polymeric material comprises a durable polymer.

23. The medical device of claim 19, wherein the polymeric material comprises a bioerodable polymer.

24. The medical device of claim 1, wherein the macrocyclic lactone is administered from the temporary device.

25. The medical device of claim 1, wherein the temporary device comprises an expandable member which comprises a coating containing the macrocyclic lactone.

26. The medical device of claim 1, wherein the medical device provides a concentration of the macrocyclic lactone or the compound in adjacent tissue from about 0.001 ng/gm tissue to about 1000 μg/gm tissue.

27. The medical device of claim 1, wherein the medical device provides a concentration of the macrocyclic lactone or the compound in adjacent tissue from about 1 ng/gm tissue to about 500 μg/gm tissue.

28. The medical device of claim 1, wherein the medical device provides a concentration of the macrocyclic lactone or the compound in adjacent tissue from about 100 ng/gm tissue to about 100 μg/gm tissue.

29. The medical device of claim 1, wherein:
the implant is a stent; and
the source comprises the macrocyclic lactone at less than about 10 μg macrocyclic lactone/mm stent, wherein the macrocyclic lactone is contained in poly(n-butylmethacrylate), such that the poly(n-butylmethacrylate) is present in a ratio of from about 1:5 to about 5:1 (w/w) to the macrocyclic lactone.

30. The medical device of claim 1, wherein:
the implant is a stent; and
the source comprises the macrocyclic lactone at less than about 10 μg macrocyclic lactone/mm stent, wherein the macrocyclic lactone is contained in poly(ethylene carbonate) or poly(L-lactide-co-glycolide) such that the poly(ethylene carbonate) or poly(L-lactide-co-glycolide) is present in a ratio of from about 1:5 to about 5:1 (w/w) to the macrocyclic lactone.

31. The medical device of claim 1, wherein:
the implant is a stent; and
the source comprises the macrocyclic lactone, such that the source is a coating on at least one surface of the stent, and the macrocyclic lactone is present at less than about 10 μg macrocyclic lactone/mm stent.

32. The medical device of claim 1, wherein:
the temporary device is a balloon; and
the source comprises the macrocyclic lactone at less than about 20 μg macrocyclic lactone/mm balloon, wherein the macrocyclic lactone is contained in poly(ethylene carbonate) or poly(L-lactide-co-glycolide), such that the poly(ethylene carbonate) or poly(L-lactide-co-glycolide) is present in a ratio of from about 1:5 to about 5:1 (w/w) to the macrocyclic lactone.

33. The medical device of claim 1, wherein:
the temporary device is a balloon;
the source comprises the macrocyclic lactone at less than about 20 μg macrocyclic lactone/mm balloon; and
the source optionally is a coating on a surface of the balloon.

34. A method of inhibiting cell proliferation, inflammation or cytokine production in a subject, comprising systemic or local administration to the subject of a therapeutically effective amount of a macrocyclic lactone of a compound of formula (I): 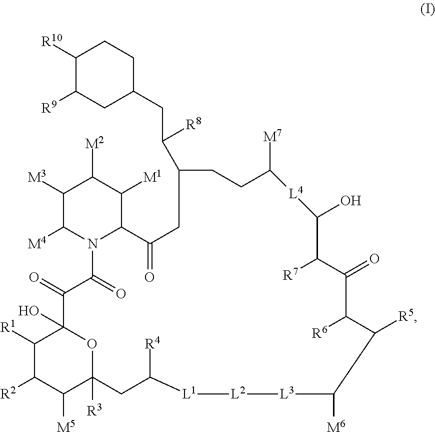
wherein:
R¹, R², R³, R⁵, R⁶, R⁸, M¹, M², M³, M⁴, M⁵, M⁶ and M⁷ are each independently H, —OH, C₁₋₆ alkyl or C₁₋₆ hydroxyalkyl;
R⁴, R⁷ and R⁹ are each independently H, —OH or C₁₋₆ alkoxy;
R¹⁰ is H, —OH, —OP(O)Me₂, 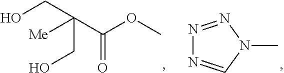
—O—(CH₂)ₙ—OH or —O—(CH₂)ₘ—O—(CH₂)ₒ—CH₃, wherein subscripts n and m are each independently from 2 to 8 and subscript o is from 1 to 6;
L¹ and L⁴ are each independently 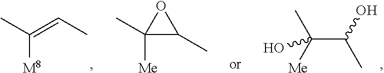
wherein each M⁸ is independently C₁₋₆ alkyl or C₁₋₆ hydroxyalkyl; and
L² and L³ are each independently 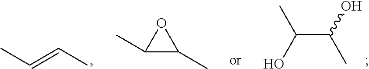
with the proviso that the macrocyclic lactone is not myolimus.

35. The method of claim 34, wherein the macrocyclic lactone is administered intravascularly, intravenously, subcutaneously, parenterally, intramuscularly, intraperitoneally, intralesionally, intrapericardially, intrathecally, intranasally, pulmonarily, mucosally, topically, transdermally, ophthalmically, or as a suppository.

36. The method of claim 34, wherein the macrocyclic lactone is administered from a temporary device or an implant.

37. The method of claim 36, wherein the temporary device is selected from the group consisting of catheters, balloons, and porous balloons.

38. The method of claim 36, wherein the implant is a luminal prosthesis.

39. The method of claim 38, wherein the luminal prosthesis comprises an expandable scaffold.

40. The method of claim 38, wherein the luminal prosthesis comprises a stent or a graft.

41. The method of claim 36, wherein the temporary device or the implant provides a concentration of the macrocyclic lactone or the compound in adjacent tissue from about 0.001 ng/gm tissue to about 1000 μg/gm tissue.

42. The method of claim 36, wherein the temporary device or the implant provides a concentration of the macrocyclic lactone or the compound in adjacent tissue from about 1 ng/gm tissue to about 500 μg/gm tissue.

43. The method of claim 36, wherein the temporary device or the implant provides a concentration of the macrocyclic lactone or the compound in adjacent tissue from about 100 ng/gm tissue to about 100 μg/gm tissue.

44. The method of claim 34, wherein the IC₅₀ of the macrocyclic lactone or the compound is from about 0.01 nM to about 1 μM.

45. The method of claim 34, wherein the IC₅₀ of the macrocyclic lactone or the compound is from about 0.1 nM to about 0.5 μM.

46. The method of claim 34, wherein the IC₅₀ of the macrocyclic lactone or the compound is from about 1 nM to about 100 nM.

47. The method of claim 34, wherein the effective total dose, or dose per administration or dose over a certain period, of the macrocyclic lactone is from about 0.1 mg to about 20 mg, or from about 0.5 mg to about 10 mg, or from about 1 mg to about 5 mg.

48. The method of claim 34, wherein the effective total dose, or dose per administration or dose over a certain period, of the macrocyclic lactone is from about 1 μg to about 1 mg, or from about 40 μg or 50 μg to about 1 mg, or from about 100 μg to about 1 mg.

49. The method of claim 34, wherein the effective total dose, or dose per administration or dose over a certain period, of the macrocyclic lactone is from about 0.5 μg or 1 μg to about 500 μg, or from about 40 μg or 50 μg to about 500 μg, or from about 20 μg to about 200 μg.

50. A method of treating an ophthalmic condition or disease in a subject, comprising administering to the subject a therapeutically effective amount of a macrocyclic lactone of a compound of formula (I): 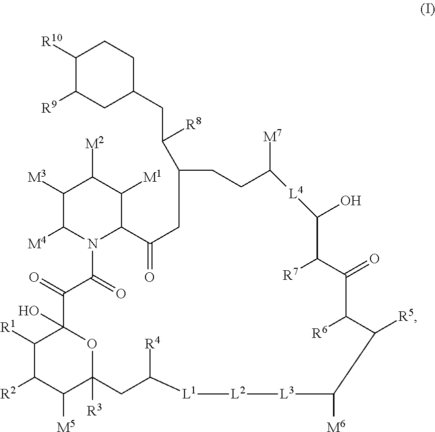
wherein:
R¹, R², R³, R⁵, R⁶, R⁸, M¹, M², M³, M⁴, M⁵, M⁶ and M⁷ are each independently H, —OH, C₁₋₆ alkyl or C₁₋₆ hydroxyalkyl;
R⁴, R⁷ and R⁹ are each independently H, —OH or C₁₋₆ alkoxy;
R¹⁰ is H, —OH, —OP(O)Me₂, 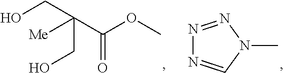
—O—(CH₂)ₙ—OH or —O—(CH₂)ₘ—O—(CH₂)ₒ—CH₃, wherein subscripts n and m are each independently from 2 to 8 and subscript o is from 1 to 6;
L¹ and L⁴ are each independently 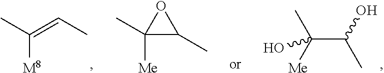
wherein each M⁸ is independently C₁₋₆ alkyl or C₁₋₆ hydroxyalkyl; and
L² and L³ are each independently 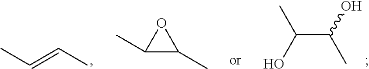
with the proviso that the macrocyclic lactone is not myolimus.

51. The method of claim 50, wherein the ophthalmic condition or disease is selected from the group consisting of disorders of the eyelid, disorders of the lacrimal system, disorders of the orbit, tear duct blockage, disorders of conjunctiva, disorders of the sclera, disorders of the cornea, disorders of the iris, disorders of the ciliary body, disorders of the lens, disorders of the choroid, disorders of the retina, Age-related Macular Degeneration (AMD), Diabetic Macular Edema (DME), glaucoma, uveitis, disorders of the vitreous body, disorders of the globe, disorders of the optic nerve, disorders of the visual pathways, disorders of the ocular muscles, disorders of binocular movement, disorders of accommodation, disorders of refraction, visual disturbances, and blindness.

52. The method of claim 50, wherein the method of treating is selected from the group consisting of inhibiting cell proliferation, inhibiting inflammation, inhibiting cytokine production, inhibiting neovascularization, inhibiting immune response, and protecting neurovascular system.

53. The method of claim 50, wherein the macrocyclic lactone is administered via an implant, an injection or an eye drop.

54. The method of claim 53, wherein the macrocyclic lactone is administered to the ocular body of the eye, the intraocular body of the eye, the vitreous body of the eye, the intravitreal body of the eye or the coroid of the eye.

55. The method of claim 53, wherein administration is via an implant.

56. The method of claim 55, wherein the macrocyclic lactone is released from the implant via osmotic pressure or diffusion.

57. The method of claim 50, wherein the macrocyclic lactone is administered with at least one additional therapeutic agent.

58. The method of claim 57, wherein the at least one additional therapeutic agent is selected from the group consisting of anti-platelet agents, anti-thrombotic agents, anti-inflammatory agents, anti-angiogenic agents, anti-proliferative agents, immunosuppressants and anti-cancer agents.

59. The method of claim 57, wherein the at least one additional therapeutic agent is selected from the group consisting of ranibizumab (Lucentis®), bevacizumab (Avastin®), pegaptanib (Macugen®), volociximab, olopatadine, mydriatcs, dexamethasone, pilocarpine, tropicamide, quinolone, galentamine, fluocinolone acetonide, triamcinolone acetonide, atropine, atropine sulfate, atropine hydrochloride, atropine methylbromide, atropine methylnitrate, atropine hyperduric, atropine N-oxide, phenylephrine, phenylephrine hydrochloride, hydroxyamphetamine, hydroxyamphetamine hydrobromide, hydroxyamphetamine hydrochloride, hydroxyamphetamine iodide, cyclopentolate, cyclopentolate hydrochloride, homatropine, homatropine hydrobromide, homatropine hydrochloride, homatropine methylbromide, scopolamine, scopolamine hydrobromide, scopolamine hydrochloride, scopolamine methylbromide, scopolamine methylnitrate, scopolamine N-oxide, tropicamide, tropicamide hydrobromide, tropicamide hydrochloride, pilocarpine, isopilocarpine, valdecoxib, celecoxib, rofecoxib, diclofenac, etodolac, meloxicam, nimesulfide, 6-MNA, L-743, L-337, NS-398, SC58125, ketorolac, clobetazol, physostigmine, stearyl ammonium chloride, benzyl ammonium chloride, and combinations thereof.
